(19) United States
(12) Patent Application Publication
(10) Pub. No.: US 2025/0276025 A1
(43) Pub. Date: Sep. 4, 2025

(54) APPLICATION OF BIFIDOBACTERIUM ANIMALIS IN IMPROVEMENT OF TUMOR IMMUNOTHERAPY RESPONSE

(71) Applicant: SHENZHEN JUNTUO BIOTECHNOLOGY CO., LTD, Shenzhen (CN)

(72) Inventors: Yan TAN, Shenzhen (CN); Yiming YIN, Shenzhen (CN); Xiaojun LIU, Shenzhen (CN); Lingyu ZHANG, Shenzhen (CN)

(73) Assignee: SHENZHEN JUNTUO BIOTECHNOLOGY CO., LTD, Shenzhen (CN)

(21) Appl. No.: 18/267,461

(22) PCT Filed: Dec. 14, 2021
(86) PCT No.: PCT/CN2021/137960

§ 371 Date: Jun. 15, 2023

(30) Foreign Application Priority Data
Dec. 15, 2020 (CN) 202011476620.4

(51) Int. Cl. A61K 35/745; A61K 35/00; A61K 39/00; A61K 39/395; A61P 35/00

(52) U.S. Cl. CPC A61K 35/745; A61K 39/39558; A61P 35/00; A61K 2035/115; A61K 2039/505

(57) ABSTRACT

The present disclosure relates to use of a probiotic composition in the manufacture of a drug for tumor treatment, the probiotic including Bifidobacterium animalis subsp. lactis. The drug further includes one or more tumor treatment drugs, such as an immune signaling pathway regulator PD-1/PDL-1. The drug is used for tumor immunotherapy.

### CROSS REFERENCE TO THE RELATED APPLICATIONS

This application is the national phase entry of International Application No. PCT/CN2021/137960, filed on Dec. 14, 2021, which is based upon and claims priority to Chinese Patent Application No. 202011476620.4, filed on Dec. 15, 2020, the entire contents of which are incorporated herein by reference.

### TECHNICAL FIELD

The present disclosure relates to the field of biomedicine, and more particularly, to use of microorganisms to improve tumor immunotherapy response.

### BACKGROUND ART

Cancer is a malignant disease with a very high mortality rate, which is difficult to treat and has a high mortality rate, resulting in a heavy burden to patients and their families. In recent years, the number of cancer patients worldwide has surged, and the latest data shows that there were 24.5 million new cancer cases and 9.6 million cancer death cases worldwide in 2017. The top 10 cancers worldwide in terms of global incidence rate are non-melanoma skin cancer (NMSC), TBL (tracheal, bronchus and lung) cancers, breast cancer, colorectal cancer, prostatic cancer, gastric cancer, liver cancer, cervical cancer, non-Hodgkin lymphoma and bladder cancer, respectively. The top 10 cancers worldwide in terms of mortality rate are TBL cancer, colorectal cancer, gastric cancer, liver cancer, breast cancer, pancreatic cancer, esophageal cancer, prostatic cancer, cervical cancer, and non-Hodgkin's lymphoma.

Colorectal cancer (CRC), also known as a large intestine cancer, rectum cancer, large intestine and rectum cancer, colon and rectum cancer, or intestinal cancer, is a cancer originating from the colon or rectum (which is a part of the large intestine). The colorectal cancer is one of the most common malignant tumors worldwide because of abnormal growth of cells that can invade or metastasize to other parts of the body. As reported by the World Health Organization (WHO), the colorectal cancer is the third most common malignant tumor in men and the second in women. According to the latest data, CRC currently ranks 4th worldwide in terms of incidence rate and 2nd in terms of mortality rate.

PD-1 (programmed death receptor 1) is a class of immune checkpoint molecules expressed by T cells and is of a member of the CD28 superfamily. PD-1 is a class of important immunosuppressive molecules that function as an “off switch” for inhibiting T cells from attacking other cells in the body. When PD-1 on the surfaces of T cells interacts with a PD-1 ligand PD-L1 (programmed death ligand-1) expressed on normal cells in vivo, cell-killing effect of the T cells is inhibited. Tumor cells use this mechanism to evade immune attack from the T cells, and express large amounts of PD-L1 to bind to PD-1 on the surfaces of the T cells and inhibit their cell-killing effect. Inhibitors targeting PD-1 or PD-L1 immune checkpoints, such as monoclonal antibody drugs, can block the binding of PD-1 to PD-L1 and inhibit its downstream signaling, thereby enhancing an immune killing effect of the T cells on the tumor cells.

However, immune checkpoint inhibitors represented by PD-1/PD-L1 inhibitors also have many problems in cancer treatment, among which a low response rate is the most prominent. Studies have shown that a response rate of patients treated with drugs targeting PD-1/PD-L1 usually does not exceed 40%, while a response rate of patients treated with CTLA-4 monoclonal antibody-ipilimumab is only about 15%, and some of the patients have only a partial response. In addition, such treatments also have the following problems: slow onset of action, with a median onset time of 12 weeks, which may delay the patient's rescuing time; poor treatment effect in some patients; side effects in patients, such as colitis, diarrhea, dermatitis, hepatitis, endocrine diseases and other immune-related adverse events (irAEs), which may lead to early termination of treatment; and high price, making it unaffordable for ordinary patients.

Bifidobacterium animalis subsp. lactis is a Gram-positive, non-motile, non-spore-forming, irregular rod-shaped anaerobic bacterium that ferments a wide range of sugars, but not starch. An optimal growth temperature is 39-42° C.

Bifidobacterium animalis subsp. lactis NexStrain 02 is isolated from the feces of healthy children. Preliminary studies show that it has a probiotic function for relieving constipation as well as acute and chronic diarrhea. This bacterium was preserved in the General Microbiological Center of the China Microbiological Culture Collection Management Committee on Jul. 27, 2020, with an Accession number of CGMCC No. 20455.

Bifidobacterium animalis subsp. lactis NexStrain 02 is continuously passaged for 100 generations, and DNA is extracted from A0, A50 and A100 generations for whole genome sequencing and analysis. The results show that MUMi values of the whole genome sequences of the A0, A50 and A100 generations are 0, indicating that the sequence of this bacterium was highly conserved.

### SUMMARY OF THE INVENTION

In order to solve the above technical problems, an object of the present disclosure is to provide a microbial agent, and a combination of the microbial agent and an immune signaling pathway regulator, are used for the treatment of tumor patients to improve the tumor response to immunotherapy.

Specifically, the present disclosure provides the following technical solutions.

In an aspect, the present disclosure provides use of a probiotic in the manufacture of a drug or kit for tumor treatment, the probiotic including Bifidobacterium animalis subsp. lactis.

According to the preceding use, the probiotic includes Bifidobacterium animalis subsp. lactis NexStrain 02 (Accession number: CGMCC No. 20455).

According to the preceding use, the probiotic includes a bacterium with an evolutionary distance of less than 0.00004 from the Bifidobacterium animalis subsp. lactis NexStrain 02.

According to the preceding use, the probiotic includes a bacterium with an evolutionary distance of less than 0.00003 from the Bifidobacterium animalis subsp. lactis NexStrain 02.

According to the preceding use, the probiotic includes a bacterium with an evolutionary distance of less than 0.00002 from the Bifidobacterium animalis subsp. lactis NexStrain 02.

According to the preceding use, the probiotic includes a bacterium with an evolutionary distance of less than 0.00001 from the Bifidobacterium animalis subsp. lactis NexStrain 02.

According to the preceding use, the probiotic includes a bacterium with an evolutionary distance of 0 from the Bifidobacterium animalis subsp. lactis NexStrain 02.

According to the preceding use, maximal unique match (MUM) between two genomes is calculated from the evolutionary distance by using a suffix tree algorithm of parsnp software, MUMi is calculated using MUM, and a value of the MUMi is a distance between the two genomes; and a MUMi calculation method is:

$$MUMi = 1 - {L}_{mum} / {L}_{av} ,$$

wherein the Lₘᵤₘ refers to a base number of all MUMs, and Lₐᵥ refers to a mean of the base numbers of the two genomes.

According to any of the preceding uses, the treatment further includes an application of one or more other tumor treatment drugs; preferably, the tumor treatment drug is an immune signaling pathway regulator; preferably, the immune signaling pathway regulator is one or more reagents selected from PD-1/PDL-1, PD-1/PDL-2, CD28/B7-1 (CD80), CD28/B7-2 (CD86), CTLA4/B7-1 (CD80), CILA4/B7-2 (CD86), 4-1BB (CD137)/4-1BBL (CD137L), ICOS/B7RP1, CD40/CD40L, Herpesvirus entry mediator (HVEM)/B- and T-lymphocyte attenuator (BTLA), OX40/OX40L, CD27/CD70, GITR/GITRL, KIR/MHC, a lymphocyte activation gene 3 (LAG3 or CD223)/MHC, a mucin domain of TIM3/TIM3 ligand, a T cell immune receptor with Ig and ITIM domains (TIGIT)/CD96, and TIGIT/CD226; and more preferably, the immune signaling pathway regulator is a PD-1 inhibitor, preferably a PD-1 antibody.

According to any of the preceding uses, the tumor is a digestive tract tumor, preferably a colorectal cancer.

According to any of the preceding uses, the tumor is a breast tumor, preferably a breast cancer.

According to any of the preceding uses, the drug or kit is used for tumor immunotherapy; preferably, the drug or kit can improve a therapy response of a patient to the PD-1 inhibitor; more preferably, the drug or kit can prolong the patient's survival time, inhibit the tumor growth, and/or improve the enrichment of anti-tumor immune cells at a tumor site; and preferably, the anti-tumor immune cells are selected from one or more of CD4+CD62L-CD44+, CD8+IFN-gamma+, CD4+TNF-alpha+, CD11c+CD86+, and/or Gr-1+CD86+.

According to any of the preceding uses, the drug or kit can simultaneously improve the therapy response of the patient to the PD-1 inhibitor, prolong the patient's survival time, inhibit the tumor growth, and improve the enrichment of anti-tumor immune cells at the tumor site; the anti-tumor immune cells include CD4+CD62L-CD44⁺, CD8⁺IFN-gamma+, CD4+TNF⁻alpha+, CD11c+CD86+ and Gr-1+CD86+; and the drug or kit can improve the enrichment of anti-tumor immune cells CD8+IFN-gamma+, CD4+TNF-alpha+, CD11c+CD86+ and Gr-1+CD86+ at the tumor site compared with the treatment with the immune signaling pathway regulator alone.

According to any of the preceding uses, a single dosage of the probiotic is at least 1-20×10⁹ CFU, or at least 2-18×10⁹ CFU, or at least 3-15×10⁹ CFU, or at least 4-12×10⁹ CFU; and preferably, the single dosage of the probiotic is at least 5-10×10⁹ CFU.

According to any of the preceding uses, the drug or kit is an oral preparation, preferably one or more of lyophilized powder, tablets, capsules, solution, and suspension.

According to any of the preceding uses, the probiotic and the other tumor treatment drug are used at the same time.

According to any of the preceding uses, the probiotic and the other tumor treatment drug are used asynchronously.

The method of the present disclosure uses the Bifidobacterium animalis subsp. lactis to improve the response to the immunotherapy of digestive tract tumors, improve the patient's survival rate, and prolong the survival time. The oral treatment method is simple and non-invasive, and basically does not cause any additional harm to the patient's body. The fermentation and lyophilization process of the Bifidobacterium animalis subsp. lactis powder is mature and stable and can be used for mass production.

### BRIEF DESCRIPTION OF THE DRAWINGS

FIG. 1 is a flowchart of an experimental design of a colon cancer CT26 model of a mouse in the present disclosure.

FIG. 2 shows tumor growth curves of respective groups of a colon cancer CT26 model of mice in Example 1 of the present disclosure.

FIG. 3 shows Kaplan-Meier survival curves of respective group of the colon cancer CT26 model of mice in Example 1 of the present disclosure.

FIG. 4 shows tumor growth curves of respective groups of a breast cancer 4T1 model of mice in Example 2 of the present disclosure.

FIG. 5 shows a comparison of CD4+CD62L+CD44+ expression quantities in respective groups for cell flow cytometry in Example 3 of the present disclosure.

FIG. 6 shows a comparison of CD4+TNF-alpha+ expression quantities in the respective groups for cell flow cytometry in Example 3 of the present disclosure.

FIG. 7 shows a comparison of CD8+IFNgamma+ expression quantities in the respective groups for cell flow cytometry in Example 3 of the present disclosure.

FIG. 8 shows a comparison of Gr-1+CD86+ expression quantities in the respective groups for cell flow cytometry in Example 3 of the present disclosure.

FIG. 9 shows tumor growth curves of respective groups of a colon cancer CT26 model of mice in Example 4 of the present disclosure.

FIG. 10 shows Kaplan-Meier survival curves of respective group of the colon cancer CT26 model of mice in Example 4 of the present disclosure.

FIG. 11 shows an effect of a NexStrain 02 culture supernatant on THP-1 cells to express IL-6 in Example 5 of the present disclosure.

FIG. 12 shows an effect of a NexStrain 02 living bacterium on THP-1 cells to express TL-6 in Example 5 of the present disclosure.

### DETAILED DESCRIPTION OF THE INVENTION

According to the above content of the present disclosure, as well as the general technical knowledge and customary means in the art, other forms of modifications, replacements or alterations may also be made without departing from the above basic technical ideas of the present disclosure.

### I. DEFINITIONS

### Immune Signaling Pathway Regulators

An immune cell messaging pathway may be regulated by one or more exemplary receptors/ligand pairs on cells: PD1/PDL1, PD1/PDL2, CD28/B7-1 (CD80), CD28/B7-2 (CD86), CTLA4/B7-1 (CD80), CILA4/B7-2 (CD86), 4-1BB (CD137)/4-1BBL (CD137L), ICOS/B7RP1, CD40/CD40L, Herpesvirus entry mediator (HVEM)/B- and T-lymphocyte attenuator (BTLA); OX40/OX40L, CD27/CD70, GITR/GITRL, KR/MIC, lymphocyte activation gene 3 (LAG3 or CD223)/MHC, mucin domain of Hepatitis A virus cellular receptor 2 (HAVCR2; also known as T cell immunoglobulin domain and mucin domain-3 (TIM3))/TIM3 ligand, T cell immune receptor with Ig and ITIM domains (TIGIT)/CD96, and TIGIT/CD226. The immune cell messaging pathway may also be regulated by one or more of the following exemplary cytokines/chemical activins and their homologous cell surface receptors: interleukin 2 (IL-2)/CD122, adenosine A2A receptor (A2AR), interleukin 6 (IL-6)/IL6R (CD126), interleukin 10 (IL-10)/IL-10R, interleukin 15 (IL-15)/IL-15R, transforming growth factor β (TGFβ)/TGFβR, and macrophage community stimulating factor 1 (CSF-1)/CSF-1R. Other immune molecules include, but are not limited to, KIR2DL, VISTA, HLLA2, TLIA, DNAM-1, CEACAM1, CD155, and indoleamine 2,3-dioxygenase (IDO), such as IDO1. Any of the above immune molecules may be targeted for the anticancer therapy as described in the present disclosure.

As used in the present disclosure, the “immune signaling pathway regulator” means a reagent that alters the activity of an immune molecule (e.g., any one of the immune molecules described in the present disclosure) relative to a control carrier. The term “regulator” is used in the broadest sense in the present disclosure and includes any molecule that partially or completely alters a messaging pathway regulated by one or more immune molecules, including a messaging pathway regulated by the molecules as described in the present disclosure.

In some cases, the immune signaling pathway regulator is an inhibitor of an immune checkpoint molecule that can reduce, slow down, stop, and/or block the activity regulated by this checkpoint molecule. The term “inhibitor” is used in the broadest sense of the present disclosure and includes any molecule that partially or completely blocks, inhibits, or neutralizes messaging pathways regulated by one or more immune checkpoint molecules, including a messaging pathway regulated by the molecules as described in the present disclosure. Suitable inhibitory molecules include natural polypeptides, peptides, antisense oligonucleotides, small organic molecules, recombinant proteins or peptides and other antagonist antibodies or antibody fragments, segments or amino acid sequence variants.

In other cases, the immune signaling pathway regulator is an activator for immune molecules that enhances and improves the activity regulated by the immune molecules. The term “activator” is used in the broadest sense in the present disclosure and includes any molecule that partially or completely alters a messaging pathway regulated by one or more immune molecules, including a messaging pathway regulated by the molecules as described in the present disclosure. Suitable activators include agonist antibodies or antibody fragments, small organic molecules, recombinant proteins or peptides. In some cases, the activator may be an agonist antibody of immune molecules, such as MEDI0562 (humanized OX40 agonist antibody), MEDI6469 (mouse OX4 agonist); and MEDI6383 (OX40 agonist).

The method of identifying such a regulator is well known in the art. For example, a candidate regulator may be brought into contact with a suitable immune molecule target, and the intensity of messaging regulated by this immune molecule may be measured by conventional assays. In the presence of a candidate regulator, detectable changes in messaging relative to a blank control indicate that the candidate regulator has the regulatory activity of an immune molecule.

### Drugs or Pharmaceutical Compositions

A bacterial solution of healthy human intestinal flora described in the present disclosure, together with one or more excipients such as adjuvants, carriers or diluents, may be placed in the form of pharmaceutical compositions, unit dosages or dosage forms. The pharmaceutical composition may be adopted in a solid dosage form (e.g., powder, granules, pellets, coated or uncoated tablets or filled capsules) or a liquid dosage form (e.g., solution, suspension, emulsion or capsules filled with the same) or a semi-solid dosage form (e.g., gel, cream and ointment). The dissolution and release properties of one or more active ingredients in pharmaceutical dosage forms may vary from seconds to months.

The “drug” or “pharmaceutical composition” is designed for use in animals and humans and may be administered by all administration routes. Preferred administration routes are an oral administration route, a pulmonary administration route, a nasal administration route, a rectal administration route, a parenteral administration route. Such pharmaceutical compositions and their unit dosage forms may routinely or in special proportions include conventional or new components, with or without additional active compounds or components, and such unit dosage forms may include any suitable effective amount of active ingredients to be adopted, commensurate with a target daily dose range.

The term “carrier” applied to the pharmaceutical composition of the present disclosure relates to a diluent, accessory or excipient administered together with an active compound.

The drug or pharmaceutical composition of the present disclosure may be administered orally, locally, parenterally, or mucosally (e.g., sublingually, by inhalation or rectally) in a dosage unit formulation containing a conventional non-toxic pharmaceutically acceptable carrier. The oral administration route is usually desired. The active reagent may be administered orally in capsules, tablets, etc.

For oral administration in the form of tablets or capsules, active pharmaceutical components may be combined with non-toxic, pharmaceutically acceptable accessories such as binders (e.g., pregelatinized cornstarch, polyvinylpyrrolidone, or hydroxypropyl methylcellulose); fillers (e.g., lactose, sucrose, glucose, mannitol, sorbitol and other reducing and non-reducing carbohydrates, microcrystalline cellulose, calcium sulfate, or dicalcium phosphate); a lubricant (e.g., magnesium stearate, talcum powder or silica, stearic acid, sodium stearyl fumarate, glyceryl docosanoate, calcium stearate, etc.); a disintegrant (e.g., potato starch or sodium starch glycolate); or a wetting agent (e.g., sodium lauryl sulfate), a colorant and flavor agent, gelatin, a sweetener, natural and synthetic gums (e.g., gum arabic, tragacanth gum or alginate), a buffer salt, carboxymethyl cellulose, polyethylene glycol, wax, etc. For oral administration in the form of liquid, the pharmaceutical components may be combined with non-toxic, pharmaceutically acceptable inert carriers (e.g., ethanol, glycerin, water), an anti-settling agent (e.g., sorbitol syrup, cellulose derivative, or hydrogenated edible fat), an emulsifier (e.g., lecithin or gum arabic), a non-aqueous carrier (e.g., almond oil, oleasters, ethanol, or fractionated vegetable oils), a preserving agent (e.g., methyl p-hydroxybenzoate or propyl p-hydroxybenzoate or sorbic acid), and the like. Stabilizers such as antioxidants (BHA, BHT, propyl gallate, sodium ascorbate, citric acid) may also be added to stabilize the dosage form.

Tablets containing active compounds may be coated by methods well known in the art. The composition of the present disclosure containing a compound of formula I as an active compound may also be introduced into beads, microspheres or microcapsules, for example, constructed from polyglycolic acid/lactic acid (PGLA). Preparations of liquids for oral administration may take, for example, in the form of solution, syrup, emulsion or suspension, or they may be presented as dry products reconstituted with water or other suitable accessories prior to use. Preparations for oral administration may be appropriately formulated to allow controlled or delayed release of the active compound.

The drug or pharmaceutical composition of the present disclosure may be delivered parenterally, i.e., by intravenous (i.v.), intraventricular (i.e.v.), subcutaneous (s.c.), intraperitoneal (i.p.), intramuscular (i.m.), subcutaneous (s.d.), or intradermal (i.d.) administration, by direct injection, by, for example, rapid concentrated injection or continuous infusion. Preparations for injection may be presented in the unit dosage form, for example, in ampoules or multi-dose containers with added preserving agents. The composition may take the shape of an excipient, and in the form of suspension, solution or emulsion in an oil or aqueous carrier, and may include a formulation reagent such as an anti-settling agent, a stabilizer and/or a dispersant. Alternatively, the active ingredient may be reconstituted in powder form with a suitable carrier (e.g., sterile pyrogen-free water) prior to use.

The drug or pharmaceutical composition of the present disclosure may also be formulated for rectal administration, such as a suppository or retention enema (e.g., containing a conventional suppository matrix such as cocoa butter or other glycerides).

### Combination Treatment

The present disclosure provides a combination cancer therapy using an immune signaling pathway regulator as described in the present disclosure and a bacterial solution of intestinal flora of healthy people. As used in the present disclosure, the term “combination treatment” includes the sequential administration of these agents (e.g., an immune signaling pathway regulator and a bacterial solution of healthy human intestinal flora), i.e., each of which is administered at a different time; and substantially simultaneous administration of these therapeutic agents, or at least two agents. The sequence of each reagent, or substantially simultaneous administration, may be influenced by any appropriate route, including, but not limited to, an oral route, an intravenous route, an intramuscular route, a subcutaneous route, and direct absorption through mucosal tissues. The agent may be administered by the same route or by different routes. For example, the first agent (e.g., bacterial solution of intestinal flora of healthy people) may be administrated orally and the second agent (e.g., an anti-PD1 antibody or other anti-checkpoint antibodies) may be administered intravenously. In addition, a selected combination agent may be administered intravenously, while the other combination agents may be administered orally. Alternatively, for example, two or more agents may be administered intravenously or subcutaneously.

The combination treatment may also include further administration of the agents (e.g., an immune signaling pathway regulator and a fermented composition) as described in the present disclosure in combination with other bioactive ingredients (e.g., different anti-tumor agents) and non-pharmacological therapy (e.g., surgery or radiotherapy). In a case where the combination treatment further includes radiation therapy, the radiation therapy may be performed at any suitable time, as long as a beneficial effect is obtained from the combination of a therapeutic agent and radiation therapy. For example, under appropriate circumstances, when the radiation therapy is temporarily removed from the administration of the therapeutic agent, this beneficial effect may still be achieved after several days or even several weeks.

It should be understood that any combination of the immune signaling pathway regulator with the bacterial solution as described in the present disclosure may be used in any order to treat cancers. The combination described in the present disclosure may be selected based on a number of factors, including, but not limited to, the effectiveness of inhibiting or preventing cancer progression, the effectiveness of alleviating the side effects of another drug in combination, or the effectiveness of alleviating cancer-related symptoms. For example, the combination treatment as described in the present disclosure may reduce any side effects associated with each individual member in the combination. Some examples are provided in the following tables. For example, this bacterial solution of intestinal flora of healthy people may be used daily during treatment involving the immune signaling pathway regulator (e.g., an anti-PD1 antibody).

In some embodiments, the combination of the immune signaling pathway regulator of the present disclosure and the bacterial solution described in the present disclosure is provided in the form of a kit. The kit contains the immune signaling pathway regulator, and the bacterial solution described in the present disclosure. In some cases, the kit of the present disclosure may also contain other necessary reagents. It should be understood that various components in the kit of the present disclosure may be used in any order to treat cancers.

Any combination of the immune signaling pathway regulator described in the present disclosure and the bacterial solution of the intestinal flora of healthy people may be used to treat cancers. As used in the present disclosure, the term “cancer” refers to medical conditions regulated by tumors or malignant cell populations, proliferation, or metastasis, including solid cancers and non-solid cancers. Examples of cancers include, but are not limited to, lung cancer, kidney cancer, gastric cancer, breast cancer, brain cancer, prostate cancer, hepatocellular cancer, pancreatic cancer, cervical cancer, ovarian cancer, liver cancer, bladder cancer, urethral cancer, thyroid cancer, melanoma, head and neck cancers, colon cancer, leukemia, lymphoma, skin cancer, gastric cancer, esophageal cancer, myeloma, rectal cancer, bone cancer, uterine cancer, prostate cancer, and hematologic malignancies.

The terms “subject”, “individual” and “patient” are used interchangeably in the present disclosure and refer to mammals evaluated for treatment and/or being treated. The subjects may be humans, but also other mammals, especially those that may be used as laboratory models for human diseases, such as mice, rats, rabbits, or dogs.

As used in the present disclosure, “effective amount” means an amount of each active agent (e.g., an immune signaling pathway regulator, e.g., anti-PD1 antibody or bacterial solution of intestinal flora of healthy people) necessary to impart a therapeutic effect on a subject, whether administered alone or in combination with one or more other active agents. In some specific examples, the therapeutic effect is to inhibit growth of cancer cells and/or reduce tumor burden. In some specific examples, the amount of bacterial solution of intestinal flora of healthy people is effective in enhancing the anticancer effect of the immune signaling pathway regulator. In other specific examples, the amount of bacterial solution of intestinal flora of healthy people is effective in enhancing the immunity of a subject on cancer cells. In some specific examples, the therapeutic effect is to prevent or inhibit tumor growth. In some specific examples, the therapeutic effect is to reduce side effects associated with one or more drugs. For example, the side effects of the inhibition of PD-1 pathway (e.g., fatigue, peripheral edema, chills, fever, diarrhea, nausea, abdominal pain, cough, dyspnea, rash, pruritus, leukoplakia, arthralgia, myalgia, back pain, headache, dizziness, and/or increased aspartate amino transferase (AST) may be mitigated by co-treatment with another agent (e.g., the immune signaling pathway regulator and the bacterial solution of intestinal flora of healthy people as described in the present disclosure).

As used in the present disclosure, the term “treatment” means the application or administration of a composition containing one or more active agents to a subject who suffers from a target disease or condition, symptoms of a disease/condition, or a tendency to treat the disease/condition, for a purpose to cure, treat, alleviate, mitigate, alter, remedy, improve, enhance, or affect disease, symptoms of disease, or a tendency to treat the disease or condition.

The mitigation of a target disease/condition includes delaying the development or progression of the disease or reducing the severity of the disease. The mitigation of the disease does not necessarily require treatment outcomes. As used herein, “delaying” the development of the target disease or condition means delaying, preventing, slowing, impeding, stabilizing, and/or delaying the disease progression. Such delay may be of varying lengths of time, depending on the history of the disease and/or a subject being treated. A method of “delaying” or mitigating the development of a disease, or delaying the onset of a disease, is a method for reducing the likelihood of developing one or more symptoms of the disease within a given time frame, and/or reducing the degree of symptoms within the given time frame, compared with a case where the method is not used. This comparison is usually based on clinical studies, using a plurality of subjects sufficient to give statistically significant results.

“Development” or “progression” of the disease refers to the initial manifestations and/or subsequent progression of the disease. The development of the disease may be detected and evaluated using standard clinical techniques well known in the art. However, the development also refers to a progression that may not be detectable. For the purposes of the present invention, the development or progression refers to a biological process of a symptom. “Development” includes occurrence, recurrence, and onset. As used in the present disclosure, the “onset” or “occurrence” of the target disease or condition includes an initial onset and/or recurrence.

In some specific examples, the combination of the immune signaling pathway regulator described in the present disclosure and the bacterial solution of intestinal flora of the healthy people is administered to a subject to be treated in an amount sufficient to inhibit at least 20% (e.g., 30%, 40%, 50%, 60%, 70%, 80%, 90% or more) in an active body of one or more target messaging pathways. In other specific examples, this combination is administered in an effective amount that reduces the degree of activity of one or more target antigens by at least 20% (e.g., 30%, 40%, 50%, 60%, 70%, 80%, 90% or more).

In some specific examples, the bacterial solution of intestinal flora of healthy people described in the present disclosure is administered to a subject (e.g., human cancer patient) who has accepted or is accepting anticancer therapy involving the use of immune signaling pathway regulator (e.g., inhibitor), as described in the present disclosure.

Conventional methods known to a person of ordinary skill in the art of medicine, which may be used to administer a pharmaceutical composition to a subject depending on the type of disease to be treated or a site of the disease. This composition may also be administered by other conventional routes, e.g., oral route, parenteral route, by inhalation spray, topical route, rectal route, nasal route, oral cavity, or vaginal route, or administered through an implanted reservoir. As used in the present disclosure, the term “parenteral” includes subcutaneous, intradermal, intravenous, intramuscular, intraarticular, Intra-arterial, intrasynovial, intrasternal, intrathecal, intracerebral, and intracranial injection or infusion techniques. In addition, the composition may be administered to a subject through an injectable reservoir route, e.g., using storage tank (1, 3 or 6 months) injection or biodegradable materials and methods. In some examples, the pharmaceutical composition is administered intraocularly or intravitreously.

Regardless of oral, rectal or parenteral (including intravenous and subcutaneous) route or in some cases even topical route, an active ingredient of formula I in the present disclosure, or with one or more pharmaceutically-acceptable accessories, carriers or diluents, especially and preferably in the form of their pharmaceutical compositions, may be administered to subjects (e.g., living animals (including humans)) in need thereof in an effective amount, for treating, mitigating or improving, alleviating or eliminating indications or conditions to which they are sensitive or set forth elsewhere in the present application.

### II. SPECIFIC EMBODIMENTS

The present disclosure is further elaborated below with reference to examples. The description of specific exemplary embodiments of the present disclosure is for explanatory and illustrative purposes. These descriptions are not intended to limit the present disclosure to a precise form as disclosed, and it is clear that many changes and variations may be made according to the teachings of the present application specification. The purpose of selecting and describing exemplary embodiments is to explain the particular principles of the present disclosure and their practical applications, so that a person skilled in the art can achieve and utilize various exemplary embodiments of the present disclosure and various choices and variations.

One object of the present disclosure is to provide a probiotic composition for tumor immunotherapy, the probiotic including Bifidobacterium animalis subsp. lactis.

An object of the present disclosure is also to provide a pharmaceutical composition for tumor immunotherapy, the pharmaceutical composition containing a probiotic Bifidobacterium animalis subsp. lactis, and a pharmaceutically acceptable carrier.

In a preferred embodiment, the pharmaceutical composition is an oral preparation, including but not limited to lyophilized powder, tablets, capsules, solution, suspension, etc.

In a preferred embodiment, the probiotic includes Bifidobacterium animalis subsp. lactis NexStrain 02 (Accession number: CGMCC No. 20455). The Bifidobacterium animalis subsp. lactis NexStrain 02 is continuously passaged for 100 generations, and DNA is extracted from A0, A50 and A100 generations for whole genome sequencing and analysis. The results show that an evolutionary distance between the whole genome sequences of A0, A50 and A100 generations is 0, so the sequence of this bacterium is highly conserved.

In a preferred embodiment, the probiotic includes a bacterium with an evolutionary distance of less than 0.00004, preferably less than 0.00003, or less than 0.00002, or less than 0.00001 from the Bifidobacterium animalis subsp. lactis NexStrain 02, most preferably a bacterium with an evolutionary distance of 0 from the Bifidobacterium animalis subsp. lactis NexStrain 02.

In a preferred embodiment, maximal unique match (MUM, http://europepmc.org/backend/ptpmcrender.fcgi?accid=PMC148804&blobtype=pdf) between two genomes is calculated from the evolutionary distance by using a suffix tree algorithm of parsnp software (https://harvest.readthedocs.io/en/latest/content/parsnp.html), MUMi (http://pdfs.semanticscholar.org/6904/381c6c908a6e18817f076b6f146c205856cf.pdf) is calculated by using MUM, and a value of the MUMi is a distance between the two genomes; and a MUMi calculation method is: UMi=1−Lₘᵤₘ/Lₐᵥ, wherein the Lₘᵤₘ refers to a base number of all MUMs, and Lₐᵥ refers to a mean of the base numbers of the two genomes.

In a preferred embodiment, the evolutionary distance from the Bifidobacterium animalis subsp. lactis NexStrain 02 is determined by a bacterial whole genome sequencing and analysis method.

In a preferred embodiment, the whole genome sequencing and analysis method includes the steps of bacterial genome DNA extraction and purity detection, library construction and sequencing, genome assembly, and evolutionary distance calculation.

The genomic DNA extraction and purity detection, library construction and sequencing, genome assembly, and evolutionary distance calculation may be performed by conventional methods in the art, e.g., DNA extraction by using a Sangonbacteria genomic DNA rapid extraction kit. The purity detection methods include, but are not limited to, Nanodrop or Qubit. Exemplarily, the library construction and sequencing method includes library preparation, library quality detection, sequencing and other steps. The genome assembly includes data cleaning, gene assembly and other steps, including, but not limited to use of PCR amplification, high-throughput sequencing, kmer's de Bruijn graph algorithm assembly, etc. The evolutionary distance calculation may calculate MUMi values between different strains by using, for example, parasnp software. The MUMi value is the distance between two genomes. By calculating distances between NexStrain 50 generation, NexStrain 100 generation as well as the published 82 strains of Bifidobacterium animalis and NexStrain 10 generation, the distance between NexStrain 0 generation, NexStrain 50 generation and NexStrain 100 generation is 0, A closest distance between other 82 published Bifidobacterium animalis subspecies subsp. lactis and NexStrain 0 generation is 0.00003.

In a preferred embodiment, according to any of the above compositions, a single dosage of the probiotic is at least 1-20×10⁹ CFU, or at least 2-18×10⁹ CFU, or at least 3-15×10⁹ CFU, or at least 4-12×10⁹ CFU, preferably, at least 5-10×10⁹ CFU.

In a preferred embodiment, the tumor immunotherapy further includes the use of one or more other tumor therapeutic drugs.

Preferably, the other tumor therapeutic drugs are one or more immune signaling pathway regulators.

Preferably, the immune signaling pathway regulator includes any of the immune signaling pathway regulators known in the art, including but not limited to a reagent selected from one or more of PD-1/PDL1, PD-1/PDL2, CD28/B7-1 (CD80), CD28/B7-2 (CD86), CTLA4/B7-1 (CD80), CILA4/B7-2 (CD86), 4-1BB (CD137)/4-1BBL (CD137L), ICOS/B7RP1, CD40/CD40L, Herpesvirus entry mediator (HVEM)/B- and T-lymphocyte attenuator (BTLA), OX40/OX40L, CD27/CD70, GITR/GITRL, KIR/MHC, lymphocyte activation gene 3 (LAG3 or CD223)/MHC, mucin domain of TIM3/TIM3 ligand, T cell immune receptor with Ig and ITIM domains (TIGIT)/CD96, and TIGIT/CD226. Preferably, the immune signaling pathway regulator is a PD-1 inhibitor, preferably a PD-1 antibody.

An object of the present disclosure is also to provide a kit for the treatment of cancers, including the above-mentioned probiotic composition or pharmaceutical composition, and the immune signaling pathway regulator, preferably a PD-1 inhibitor, preferably a PD-1 antibody.

An object of the present disclosures is also to provide use of the probiotic as described above in the manufacture of a drug or kit for the treatment of cancers.

In a preferred embodiment, the cancer is a digestive tract tumor.

In a more preferred embodiment, the cancer is a colorectal cancer.

In a preferred embodiment, the cancer is a breast tumor.

In a more preferred embodiment, the cancer is a breast cancer.

In a preferred embodiment, according to any of the above uses, the drug or kit is used for tumor immunotherapy.

In a preferred embodiment, according to any of the above uses, the drug or kit can improve the therapeutic response of a patient to an immune signaling pathway regulator (e.g., PD-1 inhibitor).

In a preferred embodiment, according to any of the above uses, the drug or kit can significantly prolong the patient's survival time.

In a preferred embodiment, according to any of the above uses, the drug or kit can significantly inhibit the tumor growth.

In a preferred embodiment, according to any of the above uses, the drug or kit can significantly improve the enrichment of anti-tumor immune cells at a tumor site. Preferably, the anti-tumor immune cells are selected from one or more of CD4+CD62L-CD44+, CD8+IFN-gamma+, CD4+TNF-alpha+, CD11c+CD86+ and/or Gr-1+CD86+.

Preferably, the drug or kit can significantly improve the enrichment of anti-tumor immune cells CD4+CD62L-CD44+, CD8+IFN-gamma+, CD4+TNF-alpha+ and/or Gr-1+CD86+ at the tumor site, compared with a patient without accepting any treatment.

Preferably, the drug or kit can significantly improve the enrichment of anti-tumor immune cells CD8+IFN-gamma+, CD4+TNF-alpha+, CD11c+CD86+ and/or Gr-1+CD86+ at the tumor site, compared with a patient treated with the immune signaling pathway regulator alone.

In a preferred embodiment, the drug or kit can simultaneously improve the therapy response of the patient to the immune signaling pathway regulators (e.g., PD-1 inhibitor), prolong the patient's survival time, inhibit the tumor growth, and improve the level of anti-tumor immune cells. The anti-tumor immune cells include CD4+CD62L-CD44+, CD8+IFN-gamma+, CD4+TNF-alpha+, CD11c+CD86+ and Gr-1+CD86+. The drug or kit can improve the enrichment of anti-tumor immune cells CD8+IFN-gamma+, CD4+TNF-alpha+, CD11c+CD86+ and Gr-1+CD86+ at the tumor site compared with a patent treated with the immune signaling pathway regulators alone.

In a preferred embodiment, according to any of the above uses, a single dosage of the probiotic is at least 1-20×10⁹ CFU, or at least 2-18×10⁹ CFU, or at least 3-15×10⁹ CFU, or at least 4-12×10⁹ CFU, preferably, at least 5-10×10⁹ CFU.

Optionally, the probiotic and the immune signaling pathway regulator are used at the same time.

Optionally, the probiotic and the immune signaling pathway regulator are not used at the same time. For example, the immune signaling pathway regulator is used on the second, third or fourth day, etc. after using the probiotic.

Optionally, the probiotic is used in a first time period, the immune signaling pathway mediator is used in a second time period, and the first time period and the second period do not coincide, partially coincide or completely coincide. Optionally, the probiotic in the first period and the immune signaling pathway regulator in the second period are administrated at the same frequency or different frequencies.

Optionally, the probiotic and the immune signaling pathway regulator are administrated according to same route.

Optionally, the probiotic and the immune signaling pathway regulator are administrated according to different routes.

### III. EXAMPLES

### Example 1: Bifidobacterium animalis Subsp. lactis, Alone or in Combination with a PD-1 Immunosuppressant, for the Treatment of Digestive Tract Tumors

### 1.1 Materials, Instruments and Sources

### Tested Drug:

Bifidobacterium animalis subsp. lactis (Accession No. 20455)
Packing: 20 g/pack×1 pack, 1×10¹¹ CFU/g
Storage temperature: 4° C.
Isotype control antibody rat IgG2a
Supplier: Crown Bioscience International Limited
Article No.: CVP039
Storage temperature: 4° C.
aPD-1 antibody (RMP1-14)
Supplier: Crown Bioscience International Limited
Article No.: CVP033
Storage temperature: 4° C.
Flow cytometry antibodies (Table 1)

TABLE 1
Information on antibodies used for flow cytometry

| Labeled antibodies | Fluorescent dyes | Clones | Batch number | Suppliers |
|---|---|---|---|---|
| CD45 | AF700 | 30-F11 | 560510 | BD |
| CD4 | BUV737 | GK1.5 | 564298 | BD |
| CD8 | PerCP-Cy5.5 | 53-6.7 | 100734 | BioLegend |
| CD25 | BV510 | PC61 | 102042 | BioLegend |
| CD44 | BV785 | IM7 | 103059 | BioLegend |
| CD62L | APC | MEL-14 | 104412 | BioLegend |
| PD-1 | BV711 | 29F.1.A12 | 135231 | BioLegend |
| TIM3 | BV605 | RMT3-23 | 119721 | BioLegend |
| CD11b | BUV395 | M1/70 | 563553 | BD |
| Gr-1 | BV650 | RB6-8C5 | 108441 | BioLegend |
| CD11c | PE-ef610 | N418 | 61-0114-82 | eBioscience |
| CD86 | FITC | PO3 | 105110 | BioLegend |
| CD80 | PE-Cy7 | 16-10A1 | 104734 | BioLegend |
| IFN-gamma | PE | XMG1.2 | 12-7311-82 | eBioscience |
| TNF-alpha | BV421 | MP6.XT22 | 506328 | BioLegend |
| L/D | ef780 | NA | 65-0865-14 | eBioscience |

### Reagents:

Normal saline; supplier: Anhui Shuanghe Pharmaceutical Co., Ltd.; batch number: 171005
RMPI1640; supplier: Gibco; batch number: 8118351
Fetal bovine serum; supplier: Excell; batch number: 11G271
PBS; supplier: HyClone; batch number: AE24926283
Trypsin; supplier: Gibco; batch number: 2003760

### Instruments:

Electronic balance, instrument number: T01741; manufacturer: Scout; model number: SPX222EN
Electronic balance, instrument number: TBBAL0890; manufacturer: METTLER TOLEDO; model number: AL104
Electronic balance, instrument number: TBBAL0760; manufacturer: METTLER TOLEDO; model number: AL204
Electronic balance, instrument number: TBBAL0210; manufacturer: METTLER TOLEDO; model number: XS204
Vernier caliper, instrument number: T01675; manufacturer: SYLVAC; model number: S_CAL PRO PAT

### 1.2 Experimental Methods

### 1.2.1 Antibiotic Treatment

Day-8 to Day 7: a formulation includes Ampicillin (1 mg/mL), Neomycin (10 mg/mL), Metronidazole (10 mg/mL), Vancomycin (5 mg/mL), and Amphotericin B (0.1 mg/mL). Ampicillin is added to sterile water for mice to drink, mixed with other antibiotics and then administrated by gavage at 200 μL each twice daily.

### 1.2.2 Cell Culture

CT26 cells (SIBS: Shanghai Institute of Biological Sciences, CAT #: TCM37) are cultured in an RPMI-1640 culture medium containing 10% fetal bovine serum. CT26 cells from an exponential growth phase are collected and PBS is resuspended to a suitable concentration for mouse subcutaneous tumor inoculation.

### 1.2.3 Preparation of Test Article and Control Solutions (Table 2)

TABLE 2
Preparation of test article and control solution

| Test | | | Storage after |
|---|---|---|---|
| article/control | Preparation | Concentration | preparation |
| Rat IgG2a | Take a rat IgG2a stock solution, add PBS, | 1 mg/mL | Discard |
| (isotype control) | and prepare 19.2 mL of 1 mg/mL solution | | after use |
| aPD-1 | Take an aPD-1 stock solution, add PBS, | 1 mg/mL | Discard |
| | and prepare 19.2 mL of 1 mg/mL solution | | after use |
| Probiotic | Subpackage the probiotic NexStrain 02 (2 | 2.5 × 10¹⁰ | Discard |
| NexStrain 02 | g/tube) at the first administration and store | CFU/mL | after use |
| | airtightly at 4° C., take one tube of probiotic | | |
| | for each administration, dissolve it with | | |
| | sterile normal saline, and prepare 2.5 × 10¹⁰ | | |
| | CFU/mL of bacterial solution | | |

### 1.2.4 Animal Modeling and Experimental Scheme

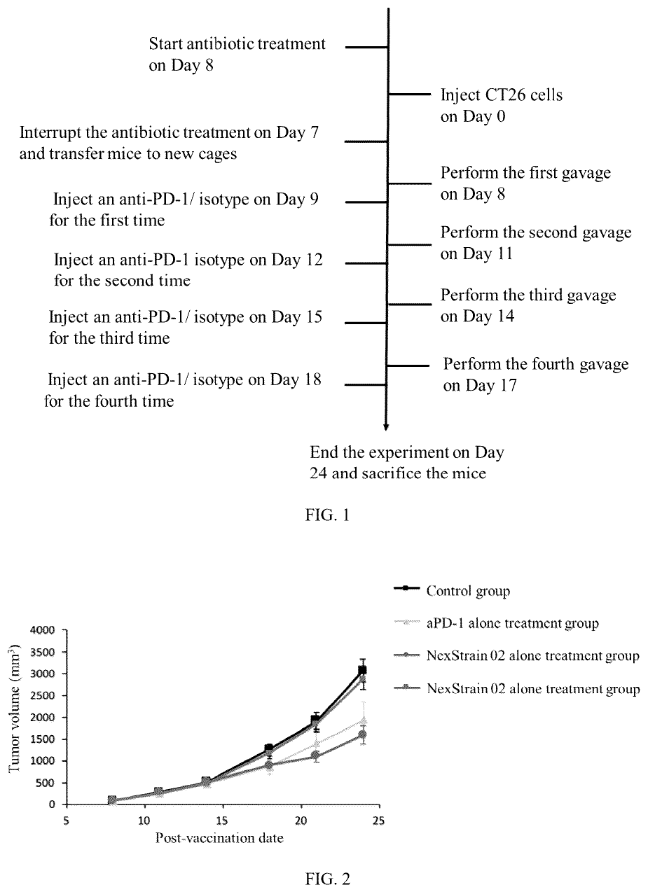

The experimental design and administration method of a CT26 model of mouse colon cancer are shown in Table 3 and FIG. 1. Specifically, SPF-graded 6- to 8-week-old female Balb/c mice are purchased.

Mice are divided into 4 groups: control group (sterile normal saline+isotype control), NexStrain 02 alone treatment group (NexStrain 02+isotype control), aPD-1 alone treatment group (sterile normal saline+aPD-1 antibody), and NexStrain 02 combination treatment group (NexStrain 02+aPD-1 antibody), with 10 mice in each group.

After the end of adaptive breeding, the mice are subjected to antibiotic treatment by gavage for 16 days (from Day−8 to Day7), with 1 gavage per day.

During antibiotic treatment, the cultured CT26 cells are injected into the mice on Day 0 for modeling, and 5×10⁵ CT26 cells are inoculated subcutaneously on the right side. The day of inoculation is defined as Day 0.

The antibiotic treatment is interrupted on Day 7 after the end of gavage and the mice are transferred to new cages to prevent the mice from eating antibiotic-containing feces. The mice are randomly grouped on Day 8 according to tumor volumes, and an average tumor volume of each group is 87 mm³.

The mice are transferred to new cages again on Day 9 before the first microbial treatment.

The mice in NexStrain 02 alone treatment group and NexStrain 02 combination treatment group are treated on Day 8, Day 11, Day 14 and Day 17, respectively, in the form of gavage, wherein each mouse is gavaged once a day with a gavage volume of 200 μL, that is, the dose is 5×10⁹ CFU/mouse/time. The mice in control group and aPD-1 alone treatment group are gavaged with an equal volume of normal saline.

The mice are injected with an antibody at an amount of 200 μg/mouse/time on Day 9, Day 12, Day 15 and Day 18, respectively. The mice in aPD-1 alone treatment group and combination treatment group are injected with an aPD-1 antibody, while the mice in control group and NexStrain 02 alone treatment group are injected with isotype control.

The mice are sacrificed on Day 24, tumors are collected, weighed, and measured for a tumor area, and tumor tissues are ground; and meanwhile, spleens are collected for flow cytometry analysis.

TABLE 3
Experimental design of anti-tumor effects of test
articles in CT26 model of mouse colon cancer

| | | Number | | | | Actual |
|---|---|---|---|---|---|---|
| | | of | Administration | Dosage | Mode of | administration |
| Nos. | Groups | animals | group | (/mice) | administration | period |
| 1 | Control | 10 | Sterile normal | — | p.o. | Days 8, 11, 14, |
| | group | | saline | | | 17 |
| | | | Rat IgG2a | 200 μg | i.p. | Days 9, 12, 15, |
| | | | | | | 18 |
| 2 | NexStrain 02 | 10 | NexStrain 02 | 5 × 10⁹ | p.o. | Days 8, 11, 14, |
| | alone | | | CFU | | 17 |
| | treatment | | Rat IgG2a | 200 μg | i.p. | Days 9, 12, 15, |
| | group | | | | | 18 |
| 3 | aPD-1 alone | 10 | Sterile normal | — | p.o. | Days 8, 11, 14, |
| | treatment | | saline | | | 17 |
| | group | | aPD-1 | 200 μg | i.p. | Days 9, 12, 15, |
| | | | | | | 18 |
| 4 | NexStrain 02 | 10 | NexStrain 02 | 5 × 10⁹ | p.o. | Day 8, 11, 14, 17 |
| | combination | | alone Treatment | CFU | | |
| | treatment | | group | | | |
| | group | | aPD-1 | 200 μg | i.p. | Days 9, 12, 15, |
| | | | | | | 18 |

### 1.2.5 Experimental Observation and Data Collection

During an experiment, the weight gain and tumor growth of mice after tumor cell inoculation are regularly monitored: tumor volume is directly measured with a vernier caliper, with a tumor volume calculation formula as follows:
Tumor volume (mm³)=½×(a×b²) (where a represents a long diameter and b represents a short diameter).

The monitoring time points are Day 5, 8, 11, 14, 17, 21 and 24; and the body weights of the mice are monitored after tumor inoculation.

A relative tumor proliferation rate, T/C %, is a percentage value of relative tumor volume or tumor weight between a treatment group and a control group at a certain time point. The calculation formula is as follows:
T/C %=T_{RTV}/C_{RTV}×100% (T_{RTV}: average RTV in treatment group; C_{RTV}: average RTV in solvent control group; RTV=Vₜ/V₀, V₀ being a tumor volume of an animal at the time of grouping, Vₜ being a tumor volume of the animal after treatment); or
T/C %=T_{TW}/C_{TW}×100% (T_{TW}: average tumor weight at the end of the experiment in treatment group; C_{TW}: average tumor weight at the end of the experiment in solvent control group).

The relative tumor suppression rate, TGI (%), is calculated as follows:
TGI %=(1−T/C)×100% (T and C are a relative tumor volume (RTV) or tumor weight (TW) in treatment group and control group at a specific time point, respectively).

On Day 13 (Day 21 after cell inoculation) after administration to all groups of mice in the CT26 model, it can be seen from the tumor growth of the mice in each group (Table 4, FIG. 2):
an average tumor volume of the mice in a sterile normal saline+isotype control group is 1916.88 mm³.
an average tumor volume of the mice in a NexStrain 02+isotype control group is 1843.34 mm³, which has no significant difference statistically compared with control group, with a p-value of 0.896 and a relative tumor growth inhibition rate TGI (%) of 2%.
an average tumor volume of the mice in a sterile normal saline+aPD-1 group is 1402.64 mm³, which has no significant difference statistically compared with control group, with a p-value of 0.159 and a relative tumor growth inhibition rate TGI (%) of 26%; and
an average tumor volume of the mice in a NexStrain 02+aPD-1 group is 1095.68 mm³, which has a significant difference statistically compared with control group, with a p-value of 0.002 and a relative tumor growth inhibition rate TGI (%) of 42%.

TABLE 4
Pharmacodynamic analysis table of each group in the CT26 model of mouse colon cancer

| | Day 13 after administration (Day 21) | | | | |
|---|---|---|---|---|---|
| | Tumor volume | Relative tumor | | | P-value |
| | mm3 | volume | TGI | T/C | (Compared with |
| Experimental group | (x̅ ± S) | (x̅ ± S) | (%) | (%) | control group) |
| Control group | 1916.88 ± 192.97 | 22.14 ± 2.08 | — | — | — |
| NexStrain 02 alone | 1843.34 ± 164.79 | 21.72 ± 2.37 | 2 | 98 | 0.896 |
| treatment group | | | | | |
| aPD-1 alone treatment | 1402.64 ± 293.17 | 16.29 ± 3.39 | 26 | 74 | 0.159 |
| group | | | | | |
| NexStrain 02 combination | 1095.68 ± 114.41 | 12.92 ± 1.43 | 42 | 58 | 0.002 |
| treatment group | | | | | |

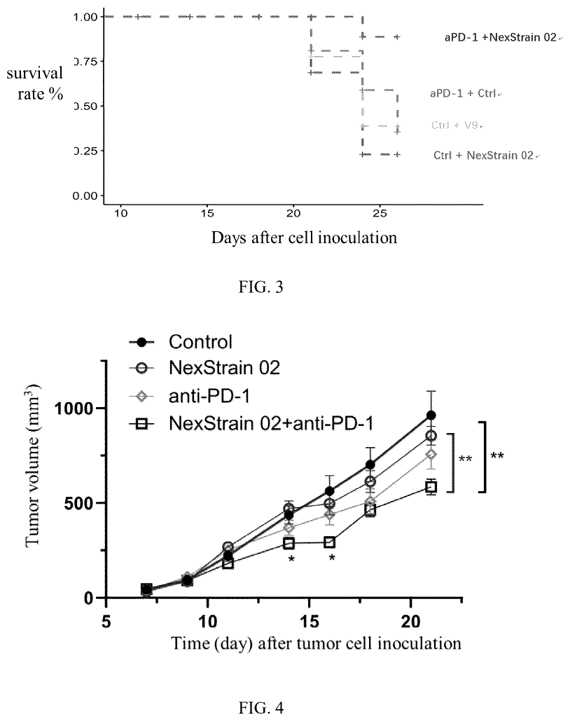

The mice in al groups of the CT26 model are subjected to survival analysis (Table 5) on Day 15 after administration (Day 23 after cell inoculation), and the survival curve of each treatment group is analyzed by a Kaplan-Meier method (FIG. 3). The NexStrain 02+aPD-1 treatment group can significantly promote the survival of the mice and improve the survival rate of the mice by 70%, which has a significant difference (p-value is 0.001) statistically compared with control group. Moreover, the NexStrain 02+aPD-1 treatment group can further improve the survival rate of the mice by 50% (p-value is 0.017) compared with control group (Table 5, FIG. 3).

TABLE 5
Survival analysis in the CT26 model of mouse colon cancer

| | Day 15 after administration (Day 24) | | | |
|---|---|---|---|---|
| | | 95% confidence | | P-value |
| | MST | interval | ILS | (Compared with |
| Experimental group | (day)* | (day) | (%)** | Group 1) |
| Control group | 21 | N/A*** | — | — |
| NexStrain 02 alone treatment | 24 | 22.141-25.859 | 14.3 | 0.531 |
| group | | | | |
| aPD-1 alone treatment group | 24 | 21.160-26.840 | 14.3 | 0.351 |
| NexStrain 02 combination | N/A | N/A | N/A | 0.001 |
| treatment group | | | | |

*MST: Median Survival Time
ILS: Increase of Life Span
***N/A: Not Applicable

### Example 2: Bifidobacterium animalis Subsp. lactis, Alone or in Combination with a PD-1 Immunosuppressant, for the Treatment of Breast Cancer

### 2.1 Experimental Reagents and Sources

TABLE 6
Reagents and sources used in breast cancer treatment experiments

| Name | Manufacturer | Article number |
|---|---|---|
| In VivoPlus anti-mouse PD-1 (CD279) | BioXcell | BP0146 |
| In VivoPlus rat IgG2a isotype control | BioXcell | BP0089 |
| Ampicillin | Sangon Biotech | G401BA0014 |
| Neomycin | Sangon Biotech | G227BA0004 |
| Metronidazole | Sangon Biotech | G608BA0022 |
| Vancomycin | Sangon Biotech | G721BA0008 |
| Amphotericin B | Selleck | S163603 |
| PBS | Solarbio | P1010-2L |

### 2.2 Experimental Methods

### 2.2.1 Animal Modeling and Experimental Scheme

The experimental design and administration method of a 4T1 model of mouse breast cancer are shown in Table 7 and FIG. 4. Specifically, SPF-graded 4- to 5-week-old female Balb/c mice are purchased.

SPF-graded healthy Balb/c mice, 120 in total, female, are purchased from Zhuhai Bestest Biotechnology Co., Ltd., License number: SCXK (Guangdong) 2020-0051; experimental unit occupancy permit number: SYXK (Guangdong) 2020-0230. All mice are grouped in cages and bred adaptively in an SPF-graded animal house for one week at room temperature (24±2)° C., alternating day and night every 12 h, and drinking water freely.

Healthy animals are selected for experiments, and the purchased Balb/c mice are adaptively bred with a basal feed for one week, during which they are observed at least once a day, and unhealthy mice are removed immediately. The animal is tagged with an ear tag method, and after sterilization and disinfection of the ear, a tag is pierced on the small ear of the mouse, so double-tag identification is realized by the mouse ear tag and the cage number.

At the end of quarantine, all mice are bred adaptively in an SPF grade for 7 days, followed by injection of breast cancer 4T1 cells at an injection dose of 105 cells/mice, an injection volume of 100 μl, and the cells are uniformly mixed before injection. The injection position is right axilla of the forelimb (near the back), and the day of cell inoculation is recorded as Day 0.

The mice inoculated with 4T1 cells are randomly divided into 4 groups, which is the same as Example 1.

The mice in NexStrain 02 alone treatment group and NexStrain 02 combination treatment group are treated on Day 0 to Day 20, respectively, in the form of gavage, wherein each mouse is gavaged once a day with a gavage volume of 200 μL, that is, the dose is 2×10⁹ CFU/mouse/time. The mice in the control group and the aPD-1 alone treatment group are gavaged with an equal volume of PBS.

The mice are injected with an antibody at an amount of 100 μg/mouse/time on Day 9, Day 12, Day 15, and Day 18, respectively. The mice in aPD-1 alone treatment group and combination treatment group are injected with an aPD-1 antibody, while the mice in control group and NexStrain 02 alone treatment group are injected with isotype control.

TABLE 7
Experimental design of anti-tumor effects of test
articles in 4T1 model of mice breast cancer

| | | Number | | | | Actual |
|---|---|---|---|---|---|---|
| | | of | Administration | Dosage | Mode of | administration |
| Nos. | Groups | animals | group | (/mice) | administration | period |
| 1 | Control group | 10 | NexStrain 02 | — | p.o. | Days 0-20 |
| | | | Mouse aPD-1 | — | i.p. | Days 9, 12, 15, |
| | | | antibody | | | 18 |
| 2 | NexStrain 02 | 10 | NexStrain 02 | 200 μL | p.o. | Days 0-20 |
| | alone treatment | | | | | |
| | group | | Mouse aPD-1 | — | i.p. | Days 9, 12, 15, |
| | | | antibody | | | 18 |
| 3 | aPD-1 alone | 10 | NexStrain 02 | — | p.o. | Days 0-20 |
| | treatment | | Mouse aPD-1 | 100 μg | i.p. | Days 9, 12, 15, |
| | group | | antibody | | | 18 |
| 4 | NexStrain 02 | 10 | NexStrain 02 | 200 μL | p.o. | Days 0-20 |
| | combination | | Mouse aPD-1 | 100 μg | i.p. | Days 9, 12, 15, |
| | treatment group | | antibody | | | 18 |

Note:
before the experiment, NexStrain 02 bacteria powder is prepared with PBS into 1*10¹⁰ CFU/mL bacterial solution, that is, the mouse gavage bacterial solution is 2*10⁹ CFU/mouse.

### 2.2.2 Experimental Observation and Data Collection

About one week after cell inoculation, on Day 7, 9, 11, 14, 16, 18 and 21, the length and width of each tumor are measured with a vernier caliper, the tumor volume is calculated, the mouse weight is weighed, and the calculation method is the same as Example 1. Grouped administration is performed according to the experimental design, with Day 21 as an experimental endpoint.

The animal's health and death are monitored daily. Routine examinations include observing the effects of the test articles and drugs on the animal's daily behavioral performances, such as behavioral activities, food intake and water intake (it is sufficient to record while changing water), weight change (weights are measured three times a week), feces shape, appearance signs, or other abnormalities.

### 2.3 Experimental Results

On Day 21 after cell inoculation of all mice in all groups of the 4T1 model, it can be seen from the tumor growth of the mice in each group: on Day 21, an average tumor volume of the mice in control group is 963.36 mm³; an average tumor volume of the mice in NexStrain 02 alone treatment group is 854.46 mm³, which has no significant difference statistically compared with control group; an average tumor volume of the mice in the aPD-1 alone treatment group is 756.32 mm³, which has no significant difference statistically compared with control group; and an average tumor volume of the mice in NexStrain 02 combination treatment group is 584.72 mm³, which has a significant difference statistically compared with control group, with a tumor growth inhibition rate of about 40% (FIG. 4).

### Example 3: Flow Cytometry (FACS)

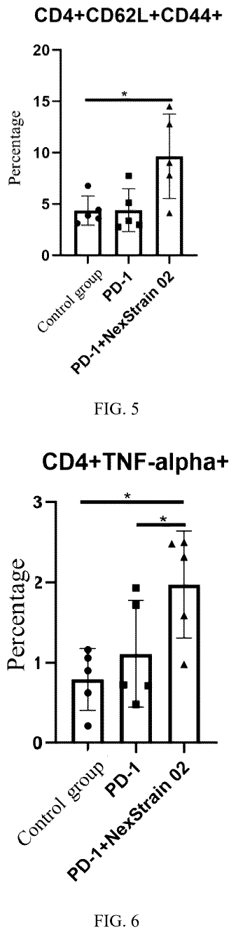

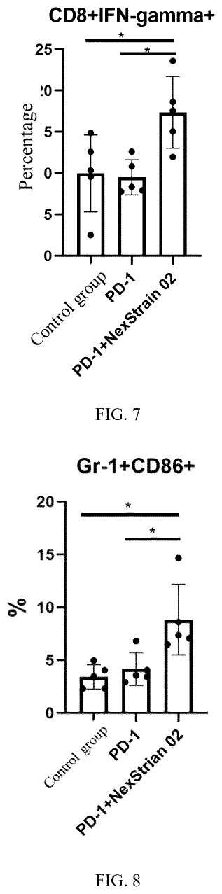

Example 1: On Day 16 after administration (Day 24 after cell inoculation), five tumors are taken from each group for FACS. After each harvested tumor sample is digested to form a single-cell suspension, BFA (calf serum) is added for blockade, and harvested for staining after 4 h of culture. The data obtained after streaming is analyzed by Kaluza software (Table 8, FIGS. 5-8), and all tests are double-tailed tests, and p-values less than 0.05 are considered statistically significant.

TABLE 8
Flow cytometry analysis results

| | | | | PD-1 alone |
|---|---|---|---|---|
| | | | Control group | treatment group |
| | | | VS | VS |
| | | | NexStrain 02 | NexStrain 02 |
| | | | combination | combination |
| Labeled antibodies | Bartlett test | ANOVA/Kruskal | treatment group | treatment group |
| CD4 + CD25+ | 0.830084993 | 0.003710242 | 0.546727783 | 0.182229793 |
| CD8 + CD25+ | 1.16E−07 | 0.547802403 | 0.547619048 | 0.69047619 |
| CD4 + CD62L − CD44+ | 0.811293388 | 0.188515713 | 0.67272369 | 0.690661726 |
| CD4 + CD62L + CD44+ | 0.004492707 | 0.007351505 | 0.031746032 | 0.055555556 |
| CD4 + CD62L + CD44+ | 0.091598039 | 0.131796808 | 0.69708748 | 0.721957243 |
| CD8 + CD62L − CD44+ | 0.184256619 | 0.419650034 | 0.328473621 | 0.849084855 |
| CD8 + CD62L + CD44+ | 0.000124406 | 0.163391236 | 0.222222222 | 0.547619048 |
| CD8 + CD62L + CD44+ | 0.003611109 | 0.2517618 | 0.841269841 | 0.841269841 |
| CD4 + IFN-gamma+ | 0.33459965 | 0.008045033 | 0.081514215 | 0.070017846 |
| CD8 + IFN-gamma+ | 0.069644912 | 0.002884079 | 0.031944943* | 0.012688435* |
| CD4 + PD-1+ | 0.306951373 | 0.001745215 | 0.950901564 | 0.137999348 |
| CD8 + PD-1+ | 0.01994163 | 0.003796311 | 0.69047619 | 0.150793651 |
| CD4 + Tim3+ | 0.587038818 | 0.003133036 | 0.279286735 | 0.214595777 |
| CD8 + Tim3+ | 0.272928077 | 0.091118213 | 0.204001626 | 0.448976224 |
| CD4 + TNF-alpha+ | 0.000106413 | 0.003309415 | 0.031746032* | 0.031746032* |
| CD8 + TNF-alpha+ | 0.640661834 | 0.645180171 | 0.336682379 | 0.471481423 |
| Gr-1 + CD80+ | 0.545307749 | 0.009189762 | 0.070760505 | 0.122908413 |
| Gr-1 + CD86+ | 0.015922663 | 0.000571872 | 0.007936508* | 0.007936508* |
| CD11c + CD80+ | 0.379361029 | 0.003868626 | 0.214183271 | 0.336071764 |
| CD11c + CD86+ | 0.001702737 | 0.015820541 | 0.055555556 | 0.015873016* |

Note:
P values less than 0.05 are indicated with an asterisk (*).

It can be seen from the above that the NexStrain 02 combination treatment group has significant differences compared with control group in the following indicators: CD4+CD62L-CD44+, CD8+IFN-gamma+, CD4+TNF-alpha+ and Cr-1+CD86+. In addition, NexStrain 02+aPD-1 treatment group has significant difference compared with the sterile normal saline+aPD-1 group in the following indicators: CD8+IFN-gamma+, CD4+TNF-alpha+, CD11c+CD86+ and Cr-1+CD86+, indicating that NexStrain 02 can promote the enrichment of T cells and dendritic cells, and improve the response of PD-1 by stimulating the immune system to improve the response of PD-1, inhibit the tumor growth, improve the survival rate, and prolong the survival time.

### Example 4: Bifidobacterium animalis Subsp. lactis Administrated Simultaneously or in Advance, Alone or in Combination with a PD-1 Immunosuppressant, for the Treatment of Digestive Tract Tumors

### 4.1 Materials, Instruments and Sources

Laboratory animals: BALB/c mice, female, 7-8 weeks (mouse age at the time of tumor cell inoculation), with a body weight 16.5-22.3 g, in a total of 84. The mice are purchased from Shanghai Lingchang Biotechnology Co., Ltd., animal certificate number: 20180003007017. Breeding environment: SPF grade.

Environmental conditions of an experimental animal breeding house: the experimental animals are bred in an independent ventilation box with constant temperature of 21-25° C. and humidity of 43-70%, ventilated at 10-20 times/hour, and alternately irradiated in light and dark for 12 h/12 h; continuously supplied with a cobalt 60 radiation-sterilized rat full-price pelleted feed for unlimited free intake, and fed with tap water (used after autoclaving) with drinking water bottles being uninterruptedly supplied with water for free ingestion. The mouse breeding box is a polysulfone mouse box, which is used after autoclaving and has a size of 325 mm×210 mm×180 mm. Autoclaved corn cobs are used as padding. 4 animals are put in each box, and a cage card is marked with IACUC approval number, experiment number, experiment start time, project leader, experimenter, animal source, group, and animal number, etc.; and the experimental animals are marked with ear tags.

### Tested Drug:

Bifidobacterium animalis subsp. lactis (Accession No. 20455)
Packing: 30 g/pack×1 pack+50 g/pack×1 pack, 1×10¹¹ CFU/g
Storage temperature: 4° C.
Isotype control antibody rat IgG2a
Supplier: Crown Bioscience International Limited
Article No.: CVP039
Storage temperature: 4° C.
aPD-1 antibody (RMP1-4)
Supplier: Crown Bioscience International Limited
Article No.: CVP033
Storage temperature: 4° C.

### Reagents

Normal saline; supplier: Jiangsu Huai'an Shuanghe Pharmaceutical Co., Ltd; batch number: 1907014C
RMPI1640; supplier: Gibco; batch number: 8119313
Fetal bovine serum; supplier: Excell; batch number: 11H305
PBS; supplier: HyClone; batch number: AE24926283
Trypsin; supplier: Gibco; batch number: 2120734

### Instruments:

Electronic balance, instrument number: T01741; manufacturer: Scout; model number: SPX222EN
Electronic balance, instrument number: TBBAL0890; manufacturer: METTLER TOLEDO; model number: XS105DU
Electronic balance, instrument number: TBBAL0760; manufacturer: METTLER TOLEDO; model number: AL204
Electronic balance, instrument number: TBBAL0210; manufacturer: METTLER TOLEDO; model number: XS204
Vernier caliper, instrument number: T01675; manufacturer: SYLVAC; model number: S_CAL PRO PAT

### 4.2 Experimental Methods

### 4.2.1 Antibiotic Treatment

Day−14 to Day 0 (Groups 1-4): a formulation includes Ampicillin (1 mg/mL), Neomycin (10 mg/mL), Metronidazole (1 mg/mL), Vancomycin (0.5 mg/mL), and Amphotericin B (0.1 mg/mL). Ampicillin is added to sterile water for mice to drink, mixed with other antibiotics and then administrated by gavage at 200 μL each once daily. The antibiotic treatment time is 14 days, and a water bottle containing the antibiotic is changed every three days. The antibiotic treatment is interrupted on Day 48 before microbial treatment and the mice are transferred to new cages to prevent the mice from eating antibiotic-containing feces. The mice are transferred to new cages again on Day 2 before the first microbial treatment to prevent the mice from eating feces with antibiotic residues.

Day −23 to Day −9 (Groups 5-6): a formulation includes Ampicillin (1 mg/mL), Neomycin (10 mg/mL), Metronidazole (1 mg/mL), Vancomycin (5 mg/mL), and Amphotericin B (0.1 mg/mL). Ampicillin is added to sterile water for mice to drink, mixed with other antibiotics and then administrated by gavage at 200 μL each once daily. The antibiotic treatment time is 14 days, and a water bottle containing the antibiotic is changed every three days. The antibiotic treatment is interrupted (Day −9) 48 h before microbial treatment and the mice are transferred to new cages to prevent the mice from eating antibiotic-containing feces. The mice are transferred to new cages again on Day 7 before the first microbial treatment to prevent the mice from eating feces with antibiotic residues.

### 4.2.2 Cell Culture

CT26 cells (SIBS: Shanghai Institute of Biological Sciences, CAT #: TCM37) are cultured in a RPMI-1640 culture medium containing 10% fetal bovine serum. CT26 cells from an exponential growth phase are collected and PBS is resuspended to a suitable concentration for mouse subcutaneous tumor inoculation.

### 4.2.3: Preparation of Test Article and Control Solution (Table 9)

TABLE 9
Preparation of test article and control solution

| Test | | | Storage after |
|---|---|---|---|
| article/control | Preparation | Concentration | preparation |
| Rat IgG2a | Take a rat IgG2a stock solution, add PBS, and | 1 mg/mL | Discard |
| (isotype control) | prepare 23.5 mL of 1 mg/mL solution | | after use |
| aPD-1 | Take an aPD-1 stock solution, add PBS, and | 1 mg/mL | Discard |
| | prepare 23.5 mL of 1 mg/mL solution | | after use |
| NexStrain 02 | Subpackage the probiotic NexStrain 02 (8 | 5 × 10¹⁰ | Discard |
| | g/tube) at the first administration and store it | CFU/mL | after use |
| | airtightly at 4° C., take one tube of probiotic for | | |
| | each administration, dissolve with sterile normal | | |
| | saline, and prepare 5 × 10¹⁰ CFU/mL of bacterial | | |
| | solution | | |

### 4.2.4 Animal Modeling and Experimental Scheme

The experimental design and administration method of a CT26 model of mouse colon cancer are shown in Table 10. Specifically, 84 SPF-graded 6- to 8-week-old female Balb/c mice are purchased and bred adaptively for one week. Mice in the same group but bred in different cages are randomly exchanged during the experiment.

Mice are divided into 6 groups: control group (sterile normal saline+isotype control), aPD-1 alone treatment group (sterile normal saline+aPD-1 antibody), probiotic NexStrain 02 alone treatment group 1 (NexStrain 02+isotype control, administrated at the same time), probiotic NexStrain 02 combination treatment group (probiotic NexStrain 02+aPD-1 antibody, administrated at the same time), probiotic NexStrain 02 alone treatment group (NexStrain 02+isotype control, administrated in advance) and probiotic NexStrain 02 combination treatment group 2 (probiotic NexStrain 02+aPD-1 antibody, administrated in advance), with 14 mice in each group.

After the end of adaptive breeding, the mice are treated with an antibiotic by gavage for 15 days (control group, aPD-1 alone treatment group, NexStrain 02 alone treatment group 1 and NexStrain 02 combination treatment group 1 from Day 14 to Day 0, and NexStrain 02 alone treatment group 2 and NexStrain 02 combination treatment group 2 from Day 23 to Day 9), and are gavaged once a day.

During antibiotic treatment, the cultured CT26 cells are injected into the mice on Day 0 for modeling, and 5×10⁵ CT26 cells are inoculated subcutaneously on the right side. The day of inoculation is defined as Day 0. The mice are randomly grouped according to mouse weights.

The mice are transferred to new cages again on before the first microbial treatment.

The mice in probiotic NexStrain 02 alone treatment group 1 and probiotic NexStrain 02 combination treatment group 1 are treated on Day 2 to Day 22, respectively, in the form of gavage, wherein each mouse is gavaged once a day with an gavage volume of 200 μL, that is, the dose is 1×10¹⁰ CFU/mouse/time. The mice in probiotic NexStrain 02 alone treatment group 2 and probiotic NexStrain 02 combination treatment group 2 are treated on Day 7 to Day 13, respectively, in the form of gavage, wherein each mouse is gavaged once a day with an gavage volume of 200 μL, that is, the dose is 1×10¹⁰ CFU/mouse/time. The mice in control group and aPD-1 alone treatment group are gavaged with an equal volume of normal saline.

The mice are injected with an antibody at a dosage of 200 μg/mouse/time on Day 9, Day 12, Day 15, and Day 18, respectively. The mice in aPD-1 alone treatment group and combination treatment group are injected with an aPD-1 antibody, while the mice in control group and NexStrain 02 alone treatment group are injected with isotype control.

The individual mouse is euthanized when the tumor volume exceeds 3000 mm³, and the entire experiment ends on Day 58 after cell inoculation.

Feces samples (about 100 mg, collected from an individual mouse) of all mice are collected 72 h after the last gavage into 1.5 mL centrifuge tubes and cryopreserved at −80° C. At the end of the experiment, all mice are collected for tumors (half of the tumors are paraffin-embedded after formalin fixation, and the other half are subjected to RNA later processing), intestinal contents (averagely sub-packaged in two tubes, frozen quickly with liquid nitrogen and cryopreserved in a −80° C. freezer), and plasma (sub-packaged into two tubes, and cryopreserved at −80° C.).

TABLE 10
Experimental design of anti-tumor effects of test
subjects in CT26 model of mouse colon cancer

| | | Number | | | | |
|---|---|---|---|---|---|---|
| | | of | Administration | Dosage | Mode of | Administration |
| Nos. | Groups | animals | group | (/mice) | administration | period |
| 1) | Blank control | 14) | Sterile normal | — | p.o. | Day 2-Day 22 |
| | group | | saline | | | |
| | | | Rat IgG2a | 200 μg | i.p. | Days 9, 12, 15, |
| | | | | | | 18 |
| 2 | aPD-1 alone | 14 | Sterile normal | — | p.o. | Day 2-Day 22 |
| | treatment group | | saline | | | |
| | | | aPD-1 | 200 μg | i.p | Days 9, 12, 15, |
| | | | | | | 18 |
| 3 | NexStrain 02 | 14 | NexStrain 02 | 1 × 10¹⁰ | p.o. | Day 2-Day 22 |
| | alone treatment | | | CFU | | |
| | group 1 | | Rat IgG2a | 200 μg | i.p | Days 9, 12, 15, |
| | | | | | | 18 |
| 4 | NexStrain 02 | 14 | NexStrain 02 | 1 × 10¹⁰ | p.o. | Day 2-Day 22 |
| | combination | | | CFU | | |
| | treatment | | aPD-1 | 200 μg | i.p. | Days 9, 12, 15, |
| | group 1 | | | | | 18 |
| 5 | NexStrain 02 | 14 | NexStrain 02 | 1 × 10¹⁰ | p.o. | Day −7 to Day |
| | alone treatment | | | CFU | | 13 |
| | group 2 | | Rat IgG2a | 200 μg | i.p. | Days 9, 12, 15, |
| | | | | | | 18 |
| 6 | NexStrain 02 | 14 | NexStrain 02 | 1 × 10¹⁰ | p.o. | Day −7 to Day |
| | combination | | | CFU | | 13 |
| | treatment | | aPD-1 | 200 μg | i.p. | Days 9, 12, 15, |
| | group 2 | | | | | 18 |

### 4.2.5 Experimental Observation and Data Collection

During the experiment, the weight gain and tumor growth of mice after tumor cell inoculation are regularly monitored: tumor volume is directly measured with a vernier caliper, with a tumor volume calculation formula as follows:
Tumor volume (mm³)=½×(a×b²) (where a represents a long diameter and b represents a short diameter).

The monitoring time points are Day 5, 8, 11, 14, 17, 21 and 24; and the body weights of the mice are monitored after tumor inoculation.

A relative tumor proliferation rate, T/C %, is a percentage value of relative tumor volume or tumor weight between a treatment group and a control group at a certain time point. A calculation formula is as follows:
T/C %=T_{RTV}/C_{RTV}×100% (T_{RTV}: average RTV in the treatment group; C_{RTV}: average RTV of a solvent control group; RTV=Vₜ/V₀, V₀ being a tumor volume of an animal at the time of grouping, Vₜ being a tumor volume of the animal after treatment); or
T/C %=T_{TW}/C_{TW}×100% (T_{TW}: average tumor weight at the end of the experiment in treatment group; C_{TW}: average tumor weight at the end of the experiment in solvent control group).

A calculation formula of the relative tumor growth inhibition rate is as follows:
TGI %=(1−T/C)×100% (T and C are a relative tumor volume (RTV) or tumor weight (TW) in treatment group and control group at a specific time point, respectively).

### 4.3 Conclusion

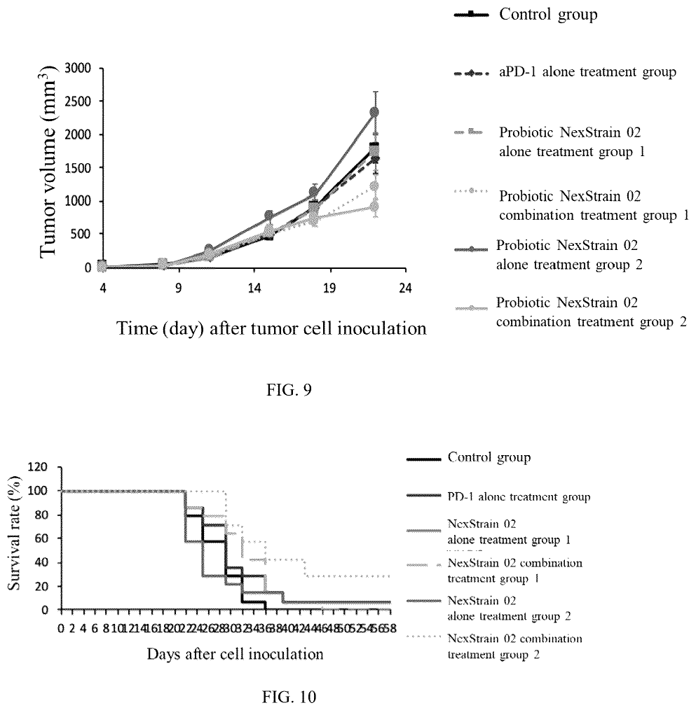

On Day 22 after the mice in all groups of the CT26 model are inoculated with cells, it can be seen from the tumor growth of the mice in each group (Table 11, FIG. 9):
an average tumor volume of the mice in control group is 1792.73 mm³;
an average tumor volume of the mice in NexStrain 02 alone treatment group 1 is 1739.35 mm³, which has no significant difference statistically compared with control group, with a p-value of 0.878 and a relative tumor growth inhibition rate TGI (%) of 3%;
an average tumor volume of the mice in NexStrain 02 alone treatment group is 1739.35 mm³, which has no significant difference statistically compared with control group, with a p-value of 0.878 and a relative tumor growth inhibition rate TGI (%) of −29%;
an average tumor volume of the mice in aPD-1 alone treatment group is 1641.84 mm³, which has no significant difference statistically compared with control group, with a p-value of 0.642 and a relative tumor growth inhibition rate TGI (%) of 8%;
an average tumor volume of the mice in NexStrain 02 combination treatment group 1 is 1206.61 mm³, which has a significant difference statistically compared with control group, with a p-value of 0.047 and a relative tumor growth inhibition rate TGI (%) of 33%; and
an average tumor volume of the mice in NexStrain 02 combination treatment group 2 is 905.21 mm³, which has a significant difference statistically compared with control group, with a p-value of 0.002 and a relative tumor growth inhibition rate TGI (%) of 50%; and

TABLE 11
Pharmacodynamic analysis table of each group
in the CT26 model of mouse colon cancer

| | Day 22 after cell inoculation | | | |
|---|---|---|---|---|
| | Tumor | | | P-value |
| | volume mm³ | TGI | T/C | (Compared |
| Experimental group | (±S) | (%) | (%) | with Group 1) |
| Control group | 1792.73 ± 215.62 | — | — | — |
| aPD-1 alone treatment group | 1641.84 ± 237.19 | 8 | 92 | 0.642 |
| NexStrain 02 alone treatment group 1 | 1739.35 ± 270.51 | 3 | 97 | 0.878 |
| NexStrain 02 combination treatment | 1206.61 ± 179.43 | 33 | 67 | 0.047 |
| group 1 | | | | |
| NexStrain 02 alone treatment group 2 | 2314.06 ± 327.01 | −29 | 129 | 0.195 |
| NexStrain 02 combination treatment | 905.21 ± 143.41 | 50 | 50 | 0.002 |
| group 2 | | | | |

Notes:
1. data are expressed as “mean ± standard error”; and
2. aPD-1 alone treatment group vs. NexStrain 02 combination treatment group 1, p = 0.155; aPD-1 alone treatment group vs. NexStrain 02 combination treatment group 2, p = 0.013.

The survival curve of each treatment group is analyzed by a Kaplan-Meier method. NexStrain 02 combination treatment group 1 and NexStrain 02 combination treatment group can significantly prolong the survival time of the mice, which has a significant difference (p-values is are 0.023 and 0.001, respectively) statistically compared with control group. In addition, compared with aPD-1 alone treatment group, NexStrain 02 combination treatment group 2 can further prolong the survival time of the mice (p=0.026). The survival of the mice in each treatment group and control group is shown in Table 12 and FIG. 10.

TABLE 12
Survival analysis in the CT26 model of mouse colon cancer

| | Day 32 after cell inoculation | | | |
|---|---|---|---|---|
| | | | | P-value |
| | MST | 95% confidence | ILS | (Comparison with |
| Experimental group | (day) | interval (day) | (%) | Group 1) |
| Control group | 29 | 25.687-32.313 | — | — |
| aPD-1 alone treatment group | 29 | 26.189-31.811 | 0 | 0.178 |
| NexStrain 02 alone treatment group 1 | 25 | 22.822-27.178 | −13.8 | 0.880 |
| NexStrain 02 combination treatment | 32 | 28.371-35.629 | 10.3 | 0.023 |
| group 1 | | | | |
| NexStrain 02 alone treatment group 2 | 25 | 22.515-27.485 | −13.8 | 0.804 |
| NexStrain 02 combination treatment | 36 | 28.742-43.258 | 24.1 | 0.001 |
| group 2 | | | | |

Note:
aPD-1 alone treatment group vs. NexStrain 02 combination treatment group 1, p = 0.700; aPD-1 alone treatment group vs. NexStrain 02 combination treatment group 2, p = 0.026.

### Example 5: Bifidobacterium animalis Subsp. lactis Promotes THP-1 Cells to Express IL-6

### 5.1 Materials, Instruments and Sources

### Cell:

THP-1 human acute monocytic leukemia cells, purchased from BNCC, having an article number: 100122.

### Reagent Consumables

RPMI Medium 1640, supplier: Solarbio, article number: 201991031
Fetal Bovine Serum, supplier: Gibco, article number: 2045512CP
1×PBS buffer, supplier: Solarbio, article number: 20200423
75 cm² Cell Culture Flask, supplier: NEST, article number: 033020AB010402B
96 well Cell Culture Plate, supplier: NEST, article number: 091419BA020914A
PMA, supplier: Sigma-Aldrich, article number: MKCL1142
LPS, Supplier: Sigma-Aldrich, article number: 039M4004V
Trypan Blue, supplier: BBI (Sangon Biotech), Article number: F902BA0025
Colombian Medium, supplier: Nissui, article number: 20200309
0.22 μm Filter Membrane, supplier: MILLEX, article number: R9EA04763
MRS broth medium, supplier: Meilunbio, article number: M0226A
Human TL-6 Valukine ELISA kit, supplier: Novus, article number: 968607
Human TL-6/TL-6 ELISA kit, supplier: Sino Biological, article number: CW13AP2403
10 mL Stripette, supplier: Corning, article number: 9220003
5 mL Stripette, supplier: Corning, article number: 03619049

### Instruments:

Anaerobic console, Trademark: ELECTROTEK, model number: AW400TG
CO₂ incubator, Trademark: ESCO, model number: CLM-170B-8-NF
Biological safety cabinet, Trademark: AIRTECH, model number: BSC-1604 II A2
Centrifuge 5430R, Trademark: Eppendorf, model number: 5430R
Centrifuge 5804, Trademark: Eppendorf, model number: 5804
Nanodrop, Trademark: Thermo, model number: NanoDrop ONE
Water purification system, Trademark: PALL, model number: Cascade I.
Sterilization pot, Trademark: ZEALWAY, model number: GF88DA
Inverted microscope, Trademark: OLYMPUS, model number: CKX 53
Medical refrigerator, Trademark: Haier, model number: HYCD-290
Constant temperature water bath, Trademark: Shanghai Yiheng, model number: HWS-24

### 5.2 Experimental Methods

### 5.2.1 THP-1 Cell Treatment

Passage and culture of cells: THP-1, which is a suspension cell, is cultured with RPMI-1640 containing 10% FBS (no antibiotic is used in the experimental process), at an inoculation density of 5×10⁵ cells/mL and sub-cultured for 48-72 h.

### 5.2.2 Effects of NexStrain 02 on THP-1 to Express IL-6

### 5.2.2.1 Effects of NexStrain 02 Culture Supernatant on THP-1

Strain culture: a strain in a strain storage tube is inoculated to a CB medium (relevant reagents are deoxygenated in advance) and incubated anaerobically for 18-24 h. The strain concentration is measured by using nanodrop, sub-inoculated once, adjusted for the density to 1×10⁸ CFU/mL, anaerobically cultured for 18-24 h, and centrifuged at 10,000 rpm for 10 min; the supernatant is taken, and filtered with a sterile filter membrane of 0.22 μm; and a filtrate is collected and stored at 4° C. for later use.

An experiment for effects of the supernatant on THP-1: THP-1 cells are polarized in advance, an old medium is removed by suction, and 200 μL of new medium is added. 4 μL of PBS is added to blank control group; 4 μL of CB medium is added to negative control group; 0.1 μg/mL of LPS is added to positive control group; and 4 μL of strain supernatant filtrate is added to experimental group. Culture is performed at 37° C. for 24 h under 5% CO₂ and 95% air. A cell culture supernatant is collected after centrifugal for 10 min under 1000 g, and IL-6 is detected using ELISA.

### 5.2.2.2 Effects of NexStrain 02 Living Bacteria on THP-1

Strain culture: a strain in a strain storage tube is inoculated to a CB medium (relevant reagents are deoxygenated in advance) and incubated anaerobically for 18-24 h. Passage inoculation is performed once at 1:100, followed by anaerobic culture for 18-24 h. The bacterial cells are washed 3 times by using PBS, and centrifuged for 5 min under 7000 g. After washing, the strain is diluted with a deoxygenated 1640 complete medium to 1×10⁷ CFU/mL and set aside.

NexStrain 02 and THP-1 are co-cultured, the THP-1 cells are polarized in advance and an old medium is removed by suction. 2 μL of PBS and 200 μL of new medium are added to blank control group; 200 μL of 0.1 μg/mL LPS new medium is added to positive control group; and 200 μL of bacterial solution is added to experimental group. Culture is performed for 24 h in an anaerobic incubator. A cell culture supernatant is collected after centrifugal for 10 min under 1000 g, and IL-6 is detected.

### 5.3 Experimental Results

### 5.3.1 Effects of NexStrain 02 Culture Supernatant on THP-1

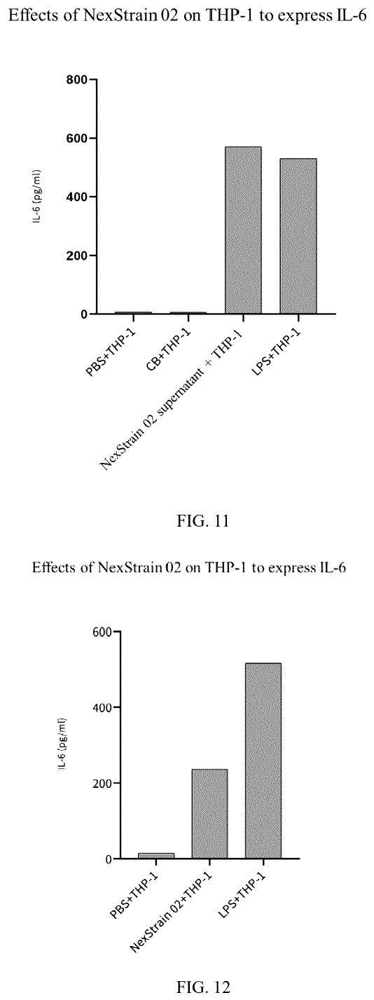

The experimental results show that the NexStrain 02 culture supernatant can promote the THP-1 cells to produce IL-6 and thus has an obvious pro-inflammatory effect (FIG. 11), revealing that NexStrain 02 may improve the PD-1 response by stimulating an immune system, inhibit the tumor growth, improve the survival rate, and prolong the survival time.

### 5.3.2 Effects of NexStrain 02 Living Bacteria on THP-1

The experimental results show that 1×10⁷ CFU/mL of NexStrain 02 can promote the THP-1 cells to produce IL-6 and thus has an obvious pro-inflammatory effect (FIG. 12), revealing that NexStrain 02 may improve the PD-1 response by stimulating an immune system, inhibit the tumor growth, improve the survival rate, and prolong the survival time.

### Example 6: NexStrain 02 Probiotic Continuous Culture Method and Genetic Stability

### 6.1 Materials, Instruments and Sources

### Test Strains

Bifidobacterium lactis NexStrain 02.

### Instruments & Equipment

Anaerobic console, biological safety cabinet, optical microscope, electronic balance, centrifuge, autoclave, PCR instrument, constant temperature incubator, etc.

### Activation Medium: TPY Medium

| Name of medium | Content (g/L) |
|---|---|
| Lactose | 10.00 |
| Beef extract | 5.00 |
| Yeast powder | 5.00 |
| Casein peptone | 10.00 |
| Soy peptone | 5.00 |
| KH₂PO₄ | 2.50 |
| K₂HPO₄ | 2.50 |
| Tween-80 | 0.25 |
| Magnesium sulfate heptahydrate | 0.1 |
| L-cysteine hydrochloride | 0.5 |

Adjust pH to 7.0, and sterilize for 15 min at 121° C. for later use

### 6.2 Test Methods

### 6.2.1 Species Activation and Deposition

Bifidobacterium lactis NexStrain 02 is removed from an −80° C. freezer, and placed in an anaerobic console to thaw, and a bacterial solution in a glycerol tube is inoculated to 10 mL of sterilized TPY liquid medium and cultured anaerobically at 37° C. for 24 h.

### 6.2.2 Plate Streaking

The activated bacterial solution is subjected to plate streaking on a TPY solid medium with an inoculation ring (note: a solid plate should be placed in the anaerobic console one day in advance for overnight deoxygenation), and the plate is inverted and cultured anaerobically at 37° C. for 72 h.

### 6.2.3 Colony Picking and Liquid Expansion Culture

Single colonies on the solid plate are picked up for microscopic examination and Gram staining to confirm strain morphology, and then the colonies are picked up and inoculated in 10 mL of TPY liquid medium for 37° C. anaerobic culture for 24 h.

### 6.2.4 Further Liquid Expansion Culture

The bacterial solution cultured in 2.3 is taken for microscopic examination and Gram staining to confirm morphological characteristics of the strain, and then inoculated into 10 mL (2% V/V) TPY liquid medium for 37° C. anaerobic culture for 24 h. Following this procedure, the bacterial solution is subjected to expansion inoculation again and inoculated into 500 mL of liquid medium for 37° C. anaerobic culture for 24 h.

### 6.2.5 Bacterial Cell Collection

The bacterial solution cultured in 2.4 is observed for Gram staining, the morphological characteristics of the strain are confirmed, and 2 mL of the bacterial solution is taken in a centrifuge tube for DNA extraction and 16S sequencing until confirmed. The remaining bacterial solution is centrifuged (4000 rpm/min, 15 min, 4° C.), the supernatant is removed, the bacterial cells are collected, added with 500 mL of sterile normal saline for washing, and then centrifuged (4000 rpm/min, 15 min, 4° C.), and the precipitate is left to complete the bacterial cell extraction.

### 6.2.6 Deposition of Strains

6.2.6.1 Protective agent formula: 10% of skimmed milk powder and 0.1% of sodium glutamate, which are sterilized at 121° C. for 7 min, quenched and cooled for later use.

6.2.6.2 Density confirmation of protective agent: a certain amount of sterilized protective agent is taken and weighed to confirm the density of the protective agent.

6.2.6.3 A cryoprotectant is added to the bacterial cells according to a ratio of wet bacterial cells to the protective agent weight (1:2), mixed well and then sub-packaged in cryopreservation tubes (500 ul per tube), and store at −80° C.

### 6.3 Strain Growth Curve Determination

The bacterial solution cultured in 2.3 is inoculated in 10 mL TPY liquid medium (set in three parallel groups) according to an inoculation amount of 1% (V/V), cultured anaerobically at 37° C. for 24 h, sampled every 2 h (samples are taken three times in each group for detection), and its absorbance value is measured at 600 nm to determine its stabilization period time.

### 6.4 Continuous Subculture of Strain

### 6.4.1 Passage Cycle

To ensure the stability of the strain, the passage should be selected in a stable period, and its passage cycle (24 passages once) should be determined according to the drawn growth curve.

### 6.4.2 Calculation of Number of Generations of Strain Culture

The strain is proliferated by dichotomy, that is, the number of bacteria after n divisions is 2n. In each culture cycle, the bacterial cells are proliferated by 100-fold, that is, the number of growth generations (i.e., the number of times the bacteria divided) in each cycle is log 2100≈6.64 generations. So, it takes 15 days to pass on 100 generations.

### 6.4.3 Initiation of Strain Subculture

After being activated for 2 generations, the original preserved strain is scribed on the solid TPY medium and cultured at 37° C. for 72 h, and then colonies are picked up for staining and microscopic observation to confirm whether the morphological characteristics of the strain are present. Single colonies are randomly picked up and inoculated in the TPY liquid medium, cultured anaerobically at 37° C. for 24 h, and then inoculated into the corresponding TPY liquid medium according to an inoculation amount of 1%. No. NexStrain 02-1.

### 6.4.4 Whole Genome Sequencing and Analysis

### 6.4.4.1 Genomic DNA Extraction and Purity Testing

Genomic DNA extraction is performed during the initial culture preservation, followed by DNA extraction from A0, A50 and A100 generations. The DNA is extracted using a Sangon bacteria genomic DNA rapid extraction kit, and the extracted DNA is tested for purity by Nanodrop (A260/A280, A260/A230).

### 6.4.4.2 Whole Genome Assembly and Analysis

A sequencing company is entrusted for library construction and sequencing: DNA samples are detected, and after the detection is qualified, a Covaris ultrasonic crusher is used to randomly break the samples, and then the entire library preparation is completed by terminal repair, addition of A-tail and sequencing linker, purification, PCR amplification and other steps. After the library is constructed, Qubit 2.0 is used for preliminary quantification and dilution of the library; and then Agilent 2100 is used to detect insertion fragments of the library, and after the size of the insertion fragment is as expected, an effective concentration of the library is accurately quantified by a Q-PCR method to ensure the quality of the library. After the library detection is qualified, NovaSeq 6000, a high-throughput sequencing platform of Illumina, is used for sequencing, with a sequencing strategy of PE150.

Cutadapt (https://github.com/marcelm/cutadapt) is used to remove an adapter sequence contained in reads on next-generation sequencing data; reads with low sequencing quality are then removed using fastp (https://github.com/OpenGene/fastp); and finally, SPAdes (https://cab.spbu.ru/software/spades/) software is used. A bacterial genome sequence is assembled based on a kmer's de Bruijn graph algorithm.

The assembled NexStrain 0-generation sequence is used as a reference sequence and is calculated for an evolutionary distance from NexStrain 50 generation, NexStrain 100 generation, and different subspecies of 82 published Bifidobacterium animalis (https://www.ncbi.nlm.nih.gov/genome/browse/#!/prokaryotes/844/) using the PARSNP software (https://harvest.readthedocs.io/en/latest/content/parsnp.html). Parsnp uses a suffix tree algorithm to calculate MUM between two genomes (http://europepmc.org/backend/ptpmcrender.fcgi?accid=PMC148804&blobtype=pdf), and then the resulting MUM is used to calculate a MUMi value (http://pdfs.semanticscholar.org/6904/381c6c908a6e18817f076b6f146c205856cf.pdf). The calculation method is: MUMi=1−Lₘᵤₘ/Lₐᵥ (Lₘᵤₘ refers to a base number of all MUMs, and Lₐᵥ refers to a mean of the base numbers of two genomes), and the MUMi value is the distance between the two genomes. By calculating distances between NexStrain 50 generation, NexStrain 100 generation as well as the published 82 strains of Bifidobacterium animalis subspecies and NexStrain 10 generation, a distance between each NexStrain 0 generation, NexStrain 50 generation and NexStrain 100 generation is 0, A closest distance between the other published 82 strains of Bifidobacterium animalis subspecies and NexStrain 0 generation is 0.00003. Exemplary data are shown in Table 13.

TABLE 13
Exemplary results of evolutionary distance determination

| | | | | | NexStrain_A0- | | | |
|---|---|---|---|---|---|---|---|---|
| | IDCC4301 | BIO5764 | BF052 | BB-12_1 | 1.ref | NexStrain_A100-1 | NexStrain_A50-1 | V9 |
| IDCC4301 | 0 | 0.00017 | 8.00E−05 | 0.00062 | 0.00016 | 0.00016 | 0.00016 | 0.00013 |
| BIO5764 | 0.00017 | 0 | 0.00011 | 0.00065 | 0.00019 | 0.00019 | 0.00019 | 0.00016 |
| BF052 | 8.00E−05 | 0.00011 | 0 | 0.00056 | 1.00E−04 | 1.00E−04 | 1.00E−04 | 7.00E−05 |
| BB-12_1 | 0.00062 | 0.00065 | 0.00056 | 0 | 0.00064 | 0.00064 | 0.00064 | 0.00061 |
| NexStrain_A0-1.ref | 0.00016 | 0.00019 | 1.00E−04 | 0.00064 | 0 | 0 | 0 | 0 |
| NexStrain_A100-1 | 0.00016 | 0.00019 | 1.00E−04 | 0.00064 | 0 | 0 | 0 | 0 |
| NexStrain_A50-1 | 0.00016 | 0.00019 | 1.00E−04 | 0.00064 | 0 | 0 | 0 | 0 |
| V9 | 0.00013 | 0.00016 | 7.00E−05 | 0.00061 | 3.00E−05 | 3.00E−05 | 3.00E−05 | 0 |

1. A method of treating a tumor, comprising administering a probiotic to a subject in need, wherein the probiotic comprises Bifidobacterium animalis subsp. lactis.

2. The method according to claim 1, wherein the probiotic comprises Bifidobacterium animalis subsp. lactis NexStrain 02 (Accession Number: CGMCC No. 20455).

3. The method according to claim 1, wherein the probiotic comprises a bacterium with an evolutionary distance of less than 0.005 from the Bifidobacterium animalis subsp. lactis NexStrain 02;
preferably, the probiotic comprises a bacterium with an evolutionary distance of less than 0.001 from the Bifidobacterium animalis subsp. lactis NexStrain 02;
preferably, the probiotic comprises a bacterium with an evolutionary distance of less than 0.0005 from the Bifidobacterium animalis subsp. lactis NexStrain 02;
preferably, the probiotic comprises a bacterium with an evolutionary distance of less than 0.00005 from the Bifidobacterium animalis subsp. lactis NexStrain 02;
preferably, the probiotic comprises a bacterium with an evolutionary distance of 0 from the Bifidobacterium animalis subsp. lactis NexStrain 02;
maximal unique match (MRUM) between two genomes is calculated from the evolutionary distance by using a suffix tree algorithm of parsnp software, MUMi is calculated by using MRUM, and a value of the MUMi is a distance between the two genomes; and a MUMi calculation method is:
$$MUMi = 1 - {L}_{mum} / {L}_{av} ,$$
wherein the Lₘᵤₘ refers to a base number of all MUMs, and Lₐᵥ refers to a mean of the base numbers of the two genomes.

4. The method according to claim 1, further comprises an application of one or more other tumor therapeutic drugs; preferably, the tumor therapeutic drug is an immune signaling pathway regulator; preferably, the immune signaling pathway regulator is one or more reagents selected from PD-1/PDL-1, PD-1/PDL-2, CD28/B7-1 (CD80), CD28/B7-2 (CD86), CTLA4/B7-1 (CD80), CILA4/B7-2 (CD86), 4-1BB (CD137)/4-1BBL (CD137L), ICOS/B7RP1, CD40/CD40L, Herpesvirus entry mediator (HVEM)/B- and T-lymphocyte attenuator (BTLA), OX40/OX40L, CD27/CD70, GITR/GITRL, KIR/MHC, lymphocyte activation gene 3 (LAG3 or CD223)/MHC, mucin domain of TIM3/TIM3 ligand, T cell immune receptor with Ig and ITIM domains (TIGIT)/CD96, TIGIT/CD226; and more preferably, the immune signaling pathway regulator is a PD-1 inhibitor, preferably a PD-1 antibody.

5. The method according to claim 1, wherein the tumor is a digestive tract tumor or breast tumor, preferably a colorectal cancer or breast cancer.

6. The method according to claim 1, wherein the method is used for tumor immunotherapy; preferably, the method can improve a therapy response of a patient to the PD-1 inhibitor; more preferably, the method can prolong patient's survival time, inhibit tumor growth, and/or improve enrichment of anti-tumor immune cells at a tumor site; and preferably, the anti-tumor immune cells are selected from one or more of CD4+CD62L-CD44+, CD8+IFN-gamma+, CD4+TNF-alpha+, CD11c+CD86+, and/or Gr-1+CD86+.

7. The method according to claim 1, wherein the method can simultaneously improve the therapy response of patient to the PD-1 inhibitor, prolong patient's survival time, inhibit tumor growth, and improve enrichment of anti-tumor immune cells at the tumor site; the anti-tumor immune cells comprise CD4+CD62L-CD44+, CD8+IFN-gamma+, CD4+TNF-alpha+, CD11c+CD86+ and Gr-1+CD86+; and the method can improve enrichment of anti-tumor immune cells CD8+IFN-gamma+, CD4+TNF-alpha+, CD11c+CD86+ and Gr-1+CD86+ at the tumor site compared with treatment with the immune signaling pathway regulators alone.

8. The method according to claim 1, wherein a single dosage of the probiotic is at least 1-20×10⁹ CFU, or at least 2-18×10⁹ CFU, or at least 3-15×10⁹ CFU, or at least 4-12×10⁹ CFU, or at least 5-6×10⁹ CFU; and preferably, the single dosage of the probiotic is at least 5-10×10⁹ CFU.

9. The method according to claim 1, wherein the probiotic is an oral preparation, preferably one or more of lyophilized powder, tablets, capsules, solution, and suspension.

10. The method according to claim 4, wherein the probiotic and the other tumor treatment drug are used or not used at the same time.

11. A pharmaceutical composition for tumor treatment, comprising a probiotic and a pharmaceutically acceptable carrier, wherein the probiotic comprises Bifidobacterium animalis subsp. lactis.

12. The pharmaceutical composition according to claim 11, wherein the probiotic comprises Bifidobacterium animalis subsp. lactis NexStrain 02 (Accession Number: CGMCC No. 20455).

13. The pharmaceutical composition according to claim 11, wherein the probiotic comprises a bacterium with an evolutionary distance of less than 0.005 from the Bifidobacterium animalis subsp. lactis NexStrain 02;
preferably, the probiotic comprises a bacterium with an evolutionary distance of less than 0.001 from the Bifidobacterium animalis subsp. lactis NexStrain 02;
preferably, the probiotic comprises a bacterium with an evolutionary distance of less than 0.0005 from the Bifidobacterium animalis subsp. lactis NexStrain 02;
preferably, the probiotic comprises a bacterium with an evolutionary distance of less than 0.00005 from the Bifidobacterium animalis subsp. lactis NexStrain 02;
preferably, the probiotic comprises a bacterium with an evolutionary distance of 0 from the Bifidobacterium animalis subsp. lactis NexStrain 02;
wherein the maximal unique match (MUM) between two genomes is calculated from the evolutionary distance by using a suffix tree algorithm of parsnp software, MUMi is calculated by using MUM, and a value of the MUMi is a distance between the two genomes; and a MUMi calculation method is:
MUMi=1−Lmum/Lav,
wherein the Lmum refers to a base number of all MUMs, and Lav refers to a mean of the base numbers of the two genomes.

14. The pharmaceutical composition according to claim 11, wherein the tumor treatment further comprising an application of one or more other tumor therapeutic drugs; preferably, the tumor therapeutic drug is an immune signaling pathway regulator; preferably, the immune signaling pathway regulator is one or more reagents selected from PD-1/PDL-1, PD-1/PDL-2, CD28/B7-1 (CD80), CD28/B7-2 (CD86), CTLA4/B7-1 (CD80), CILA4/B7-2 (CD86), 4-1BB (CD137)/4-1BBL (CD137L), ICOS/B7RP1, CD40/CD40L, Herpesvirus entry mediator (HVEM)/B- and T-lymphocyte attenuator (BTLA), OX40/OX40L, CD27/CD70, GITR/GITRL, KIR/MHC, lymphocyte activation gene 3 (LAG3 or CD223)/MHC, mucin domain of TIM3/TIM3 ligand, T cell immune receptor with Ig and ITIM domains (TIGIT)/CD96, TIGIT/CD226; and more preferably, the immune signaling pathway regulator is a PD-1 inhibitor, preferably a PD-1 antibody.

15. The pharmaceutical composition according to claim 11, wherein the tumor is a digestive tract tumor or breast tumor, preferably a colorectal cancer or breast cancer.

16. The pharmaceutical composition according to claim 11, wherein the pharmaceutical composition is used for tumor immunotherapy; preferably, the pharmaceutical composition can improve a therapy response of a patient to the PD-1 inhibitor; more preferably, the pharmaceutical composition can prolong patient's survival time, inhibit tumor growth, and/or improve enrichment of anti-tumor immune cells at a tumor site; and preferably, the anti-tumor immune cells are selected from one or more of CD4+CD62L-CD44+, CD8+IFN-gamma+, CD4+TNF-alpha+, CD11c+CD86+, and/or Gr-1+CD86+.

17. The pharmaceutical composition according to claim 11, wherein the pharmaceutical composition can simultaneously improve the therapy response of patient to the PD-1 inhibitor, prolong patient's survival time, inhibit tumor growth, and improve enrichment of anti-tumor immune cells at the tumor site; the anti-tumor immune cells comprise CD4+CD62L-CD44+, CD8+IFN-gamma+, CD4+TNF-alpha+, CD11c+CD86+ and Gr-1+CD86+; and the pharmaceutical composition can improve enrichment of anti-tumor immune cells CD8+IFN-gamma+, CD4+TNF-alpha+, CD11c+CD86+ and Gr-1+CD86+ at the tumor site compared with treatment with the immune signaling pathway regulators alone.

18. The pharmaceutical composition according to claim 11, wherein a single dosage of the probiotic is at least 1-20×10⁹ CFU, or at least 2-18×10⁹ CFU, or at least 3-15×10⁹ CFU, or at least 4-12×10⁹ CFU, or at least 5-6×10⁹ CFU; and preferably, the single dosage of the probiotic is at least 5-10×10⁹ CFU.

19. The pharmaceutical composition according to claim 11, wherein the pharmaceutical composition is an oral preparation, preferably one or more of lyophilized powder, tablets, capsules, solution, and suspension.

20. The pharmaceutical composition according to claim 14, wherein the probiotic and the other tumor therapeutic drugs are used or not used at the same time.
